(19) United States
(12) Patent Application Publication
(10) Pub. No.: US 2007/0049518 A1
(43) Pub. Date: Mar. 1, 2007

(54) Novel method of treatment of inflammatory skin conditions

(76) Inventors: Stephen Rupert Chandler, Abingdon (GB); Guy Timothy Layton, Abingdon (GB)

Correspondence Address: KLAUBER & JACKSON, 411 HACKENSACK AVENUE, HACKENSACK, NJ, 07601, US

(21) Appl. No.: 11/515,373

(22) Filed: Aug. 31, 2006

(30) Foreign Application Priority Data
Aug. 31, 2005 (GB) GB0517685.4
Jul. 14, 2006 (GB) GB0613954.7

(51) Int. Cl. A61K 38/04; A61K 38/14; A61K 31/44; A61K 31/19

(52) U.S. Cl. 514/7; 514/19; 514/575; 514/352; 514/8; 514/419

(57) ABSTRACT

There is provided, inter alia, a method for the treatment or prevention of an inflammatory skin condition which is characterised by colonisation with Staphylococcus aureus, comprising the topical administration of an aureolysin inhibitor.

### CROSS REFERENCE TO RELATED APPLICATIONS

The present application claims priority of Great Britain Application Serial No. 0517685.4 filed on Aug. 31, 2005 and Great Britain Application Serial No. 0613954.7 filed on Jul. 14, 2006. Applicants claim the benefits under 35 U.S.C. §119 as to the said Great Britain applications, and the entire disclosures of both applications are incorporated herein by reference in their entireties.

### BACKGROUND OF THE INVENTION

The present invention relates to the treatment of inflammatory skin conditions which are characterised by colonisation with Staphylococcus aureus.

Atopic dermatitis (AD), sometimes referred to as eczema, is a chronic, relapsing condition which is characterised by puritus, erythema, dry skin and inflammation.

The pathogenesis of AD is still not fully understood, although excessive T-cell activation in response to antigen stimulation and hyperstimulation of T-cells by atopic Langerhans cells are said to be important factors. Levels of IgE production correlate well to the severity of the disease, and although allergen specific IgE may be observed in many patients, it is not clear that this finding indicates sensitisation to a specific allergen.

Prevalence of the disorder varies widely, but has been estimated to be as high as 20% among children in some western countries. AD is frequently seen in families with a history of atopic diseases (asthma, allergic rhinitis and AD).

Topical application of the antibiotic mupirocin has provided significant improvements in patients with poorly controlled AD, suggesting that bacteria may be involved in the perpetuation of the disorder (Lever, R et al Br. J. Dermatol. 119:189-198, 1988).

Staphylococcus aureus has been found to colonise the skin lesions of more than 90% of AD patients (Leyden, J E, Marples, R R and Kligman A M Br. J. Dermatol 90:525-530, 1974), while being present in only 5% of normal subjects. The bacteria have been shown to be important in the exacerbation and chronicity of AD through the release of toxins (e.g. enterotoxins A, B, C and D; toxic shock syndrome toxin), many of which are highly antigenic in nature, thus exacerbating the inflammatory responses in the skin (Leung, D Y M et al J. Clin. Invest. 92 1374-80, 1993). One particular study in children found that 81% of patients had Staphylococcus aureus colonisation (compared to 4% of the control group) showing that disease severity could be correlated with colonisation by toxigenic strains (Bunikowski, R et al J. Allergy Clin Immunol. 105(4):814-819, 2000).

A unifying link between the body of evidence suggesting a role of environmental factors, including food allergens or aeroallergens, and the literature suggesting an involvement of Staphylococcus aureus colonisation was found when mice exposed to enterotoxin B and the house dust mite antigen Der p produced an additive inflammatory response (Herz, U J. Invest. Dermat. 110(3):224-231, 1999). Furthermore, Staphylococcus aureus has been shown to preferentially bind to skin sites involving Th-2 type inflammation (Cho, S-H et al J. Invest. Dermat. 116(5):658-663, 2001).

Current treatments typically involve a number of approaches (i) skin hydration—including batching and use of moisturiser (ii) the use of medicaments to reduce or modulate the immune response, such as steroids (glucocorticoids) and immunosuppressants (e.g. cyclosporine A, tacrolimus and pimecrolimus) (iii) elimination of contributory factors—irritants, allergens, emotional stress factors and infectious agents. Although current treatment can effectively deal with acute phases of the disorder, there are questions over their long-term use due to the potentially severe side effects associated with extended use of steroids and immunosuppressants. Oral antibiotics are often used to treat superinfections, although the general use of antibiotics, especially topical antibiotics is generally discouraged due to the risk of the development of antibiotic resistant bacterial strains.

Aureolysin (EC 3.4.24.29) is a metalloprotease which is secreted by Staphylococcus aureus (Dubin, G. Biol. Chem. 383:1075-1086, 2002). Aureolysin is a member of the thermolysin protein family, being dependent upon zinc and calcium for its activity, and has a low substrate specificity. The crystal structure of aureolysin was published in 1998, showing that the protein consists of a single chain of 301 amino acids (Banbula, A et al Structure 6(9):1185-1193, 1998). Aureolysin is encoded by the aur gene, genetic analysis of which indicates that the protein is highly conserved and may therefore play an important role in the lifecycle of the bacterium (Sabat, A et al Infect. Immun. 68(2):973-976, 2000). We have discovered that inhibitors of aureolysin should decrease the pathogenicity and colonising potential of Staphylococcus aureus in atopic dermatitis. This limits the virulence of the organism through perturbing aureolysin-host interactions.

The exact function of aureolysin is not clear, although it has been implicated in the processing of V8 protease (a secreted serine protease) and has been shown to inactivate the human proteinase inhibitors α₁-antichymotrypsin and α₁-proteinase inhibitor in vitro. Strains of Staphylococcus aureus which produce significant amounts of aureolysin are less susceptible to cathelicidin LL-37, a human bactericidal peptide with potent activity against Staphylococci (Sieprawska-Lupa, M et al Antimicrob. Agents Chemother. 48(12):4673-4679, 2004). However, proteolysis of anti-microbial peptides has yet to be proven as a mechanism for bacterial resistance in vivo (Brogden, K A Nature 3:238-250, 2005). Secreted toxins in general are known to be significant virulence factors, however, aureolysin is not considered to be a definite virulence factor (Supuran, C T, Scozzafava, A and Clare, B W Med. Res. Rev. 22(4):329-372, 2002; Dubin, G Biol. Chem. 383:1075-1086, 2002).

Investigation of the proteolytic activity of a range of Staphylococcus aureus strains from patients with AD found that of those strains showing moderate to high proteolytic activity, aureolysin contributed between 25-100% of the proteolytic activity (Miedzobrodzki, J et al Eur. J. Clin. Microbiol Infect. Dis. 21:269-276, 2002).

WO02/089730 discloses compounds and methods for the modulation of CD154 activity, such methods including decreasing the release of CD154 by administration of metalloprotease inhibitors in order to block the mobilisation of CD154 from the cell surface by endogenous human metalloproteases. Although primarily discussing such an approach as an anti-thrombotic therapy, treatment of AD is suggested, however there is no supportive evidence for this claim. Furthermore, neither aureolysin, nor the role of aureolysin in AD, are discussed.

Also, WO02/089730 discloses inhibition of metalloproteases but none are defined by the description or examples. The inhibitors defined are known as matrix metalloprotease, MMP (Clan MA(M), M10 family) inhibitors and 5 examples are given which are broad spectrum MMP inhibitors or MMP2/9 gelatinase inhibitors. They have, therefore, indirectly shown that MMPs can cleave CD154 but they have not defined which metalloproteases cleave CD154. The description also mentions the sheddases (adamalysin family of metalloproteases, M12 family) but does not provide evidence for their role in CD154 shedding.

Grobelny et al (1992) Biochemistry 31, 7152-7154 discusses the inhibition of human skin collagenase, thermolysin and Pseudomonas aeruginosum elastase by peptide hydroxamic acids.

A considerable number of MMPs are known in the art due to the fact that these have for a long time been of interest as drug targets in cancer and inflammatory diseases. MMPs (M10) and the adamalysin (M12) family of metalloproteases are metzincins (characterised by the ‘metzincin’ fold near the zinc binding motif (Bode et al. FEBS Lett, 331, 134-40, 1993)) and are distinct from the gluzincins (characterised by the presence of a Glu residue in the zinc binding domain) of which aureolysin (M4) is a member. Thus aureolysin is not categorised as an MMP. It has also been known for a long time that MMPs and particularly adamalysins can cleave membrane proteins releasing active molecules (e.g. TNFalpha, Fas, CD30, CD40 etc).

In summary, whereas the art has recognised a role for Staphylococcus aureus in the exacerbation of AD, proposed treatments have been directed to control of either the inflammatory response resulting from a combination of the underlying disease and Staphylococcus aureus colonisation or to control Staphylococcus aureus directly. Inhibition of a secreted protein, such as aureolysin is not envisaged in the art. Also, whereas the art has recognised a role for inhibition of some metalloproteases in the treatment of AD, this has been in the context of inhibition of endogenous matrix metalloproteases and adamalysins and not exogenous metalloproteases from colonising bacteria.

There is a clear need for new methods of treating inflammatory skin conditions which are characterised by colonisation with Staphylococcus aureus, such as AD. Furthermore, in light of the well known issues regarding the development of resistance to conventional antibacterial treatments, it is desirable that the new methods for the treatment of inflammatory skin conditions which are characterised by colonisation with Staphylococcus aureus involve a novel mechanism of action.

### SUMMARY OF THE INVENTION

According to the present invention there is provided a method for the treatment or prevention of an inflammatory skin condition in a mammal which is characterised by colonisation with Staphylococcus aureus, comprising the topical administration of an aureolysin inhibitor.

In a second aspect of the present invention there is provided the use of an aureolysin inhibitor in the manufacture of a topical medicament for the treatment or prevention of an inflammatory skin condition in a mammal which is characterised by colonisation with Staphylococcus aureus.

Also provided is a topical pharmaceutical composition comprising an aureolysin inhibitor and a pharmaceutically acceptable carrier or excipient, for use in the treatment of an inflammatory skin condition which is characterised by colonisation with Staphylococcus aureus.

The methods, uses and compositions are expected to be useful in veterinary applications (i.e. wherein the mammal is a domestic or livestock mammal e.g. cat, dog, horse, pig etc). However the principal expected use or method is in pharmaceutical applications (i.e. wherein the mammal is a human).

An advantage of the methods, uses and compositions of the invention is that in so far as the treatment targets aureolysin, since the treatment targets a secreted protein which is apparently non-critical for bacterial survival, it is much less likely to give rise to the selective pressure which can result in production of resistant mutants of the bacterium relative to approaches involving use of conventional antibiotics. Furthermore, since it targets an exogenous protein (i.e. a protein not present in mammals) then it may be expected not to result in mechanism related side effects (i.e. side effects resulting from the mechanism of inhibition rather than the inhibitory agent itself).

### BRIEF DESCRIPTION OF THE FIGURES

FIG. 1 shows gels obtained for skin wash samples taken from sites of acute AD (zymographic analysis with and without Compound 11)

FIG. 2 shows inhibition of proteolytic activity by Compound 3 in milk agar plate assay

### DETAILED DESCRIPTION OF THE INVENTION

By the term ‘an inflammatory condition which is characterised by colonisation with Staphylococcus aureus’ is meant a condition such as atopic dermatitis where the skin is colonised by Staphylococcus aureus in the majority of cases and where an increase in colonisation or cutaneous infection results in aggravation of the underlying condition and an increase in the inflammatory response.

A further inflammatory condition is Netherton's syndrome, a severe autosomal recessive skin disorder characterised by ichthyosiform erythroderma, atopy (atopic dermatitis and very high IgE levels) and trichorrhexis invaginata. Most patients experience recurrent or persistent bacterial infections.

By the term ‘atopic dermatitis’ or ‘AD’ is meant a chronic relapsing inflammatory skin disease characterised by intense pruritis and cutaneous hyperreactivity associated with elevated serum levels of IgE and eosinophils.

In a preferred embodiment, the method or use of the present invention is for the treatment or prevention of atopic dermatitis.

The aureolysin inhibitor may be any metalloprotease inhibitor which is capable of inhibiting the proteolytic activity of aureolysin. The inhibitor will preferably inhibit allelic type II and will more preferably inhibit both allelic forms, type I and type II aureolysin, type II being prevalent in skin diseases (Sabat A, Infect. Immun. 68, 973-6, 2000). This inhibitor may or may not also directly inhibit endogenous metzincin metalloproteases; for example, MMPs (M10) (e.g. MMPs 1, 2, 8, 9) and/or adamalysins (M12). Aureolysin allelic types I and II are hereinafter referred to as “aureolysin I” and “aureolysin II” respectively.

The ability of a given substance to inhibit aureolysin can be determined using the “Aureolysin enzyme inhibition assay” given in the Examples below.

By “inhibition of aureolysin” or “aureolysin inhibitor” we mean gives an IC50 value of less than 50 micromolar in the aureolysin enzyme assay (eg the aureolysin II enzyme assay), preferably less than 5 micromolar especially less than 0.5 micromolar.

By “inhibition of endogenous metzincin metalloproteases” or “endogenous metzincin metalloprotease inhibitor” we mean gives an IC50 value of less than 50 micromolar in the corresponding endogenous metzincin metalloprotease assay, preferably less than 5 micromolar especially less than 0.5 micromolar.

Thus in one embodiment of the invention the aureolysin inhibitor does not significantly inhibit endogenous metzincin metalloproteases e.g. MMP-9. By “does not significantly inhibit” is meant that the strength of inhibition (e.g. as measured by IC50) of the inhibitor against endogenous metzincin metalloproteases (e.g. MMP-9) is at least 5 times weaker preferably at least 10 times e.g. at least 50 times weaker than the strength of inhibition of the inhibitor against aureolysin (eg aureloysin II). In another embodiment of the invention the aureolysin inhibitor does significantly inhibit endogenous metzincin metalloproteases (e.g. MMP-9). By “does significantly inhibit” is meant that the strength of inhibition (e.g. as measured by IC50) of the inhibitor against endogenous metzincin metalloproteases (e.g. MMP-9) is at least 0.5 times e.g. at least 1 times the strength of inhibition of the inhibitor against aureolysin (eg aureloysin II). The strength of inhibition (e.g. as measured by IC50) of the inhibitor against endogenous metzincin metalloproteases (e.g. MMP-9) may for example be at least 10 times, perhaps 100 times or even 1000 times the strength of inhibition of the inhibitor against aureolysin (eg aureloysin II).

Known examples of endogenous MMPs are discussed in Hande et al (2004) Clinical Cancer Research 10, 909-915. These include MMP-2 (gelatinase A), MMP-9 (gelatinase B) and MMP-14 (MT-MMP-1, a membrane bound enzyme) MMP-1 (collagenase-1), MMP-3 (stromelysin-1), MMP-7 (matrilysin), MMP-11 (stromelysin-3), and MMP-13 (collagenase-3). Another example is MMP-8 (collagenase-2). Examples of adamalysins include ADAM10, ADAM17 and ADAM33. As pointed out above, a number of these enzymes have previously been linked to cancer and inflammation and ADAM33 is genetically linked to asthma.

The aureolysin inhibitor may also indirectly inhibit other tissue damaging proteases in the skin. Proteases are frequently expressed as inactive zymogens that require proteolytic cleavage to become active. Indeed, aureolysin itself is believed to be responsible for initiating the activation of the extracellular proteases secreted by Staphylococcus aureus (Shaw et al. Microbiology, 150, 217-28, 2004). Aureolysin is therefore also likely to activate endogenous host proteases present in the skin and therefore to exacerbate diseases such as AD. It is known, for example, that 3 other members of the M4 family (Pseudomonas aeruginosa elastase, Vibrio cholera proteinase and thermolysin) can activate human MMPs (Okamoto et al. J. Biol. Chem. 272, 6059-66, 1997). A closely related enzyme to aureolysin, bacillolysin, is known to activate pro-urokinase which converts plasminogen to plasmin (Narasaki et al. J. Biol. Chem. 280, 14278-87, 2005). This reference also discloses that bacillolysin converts plasminogen to a mini-plasminogen-like molecule which is more susceptible to conversion to plasmin. Our data shows that aureolysin activates pro-urokinase and inhibition of aurolysin can prevent this activation. Activation of pro-urokinase leads to the activation of the plasminogen pathway resulting in the production of pro-inflammatory plasmin. We have also shown that aureolysin activates pro-MMP-1 and inhibition of aurolysin can prevent this activation. Activation of MMP-1 leads to increased degradation of collagen in the skin, perturbing the normal skin barrier.

The aureolysin inhibitor may, for example, be selected from thermolysin inhibitors e.g. known thermolysin inhibitors; for example acyclic succinyl hydroxamates as disclosed in Marcotte et al., J. Enzyme Inhibition 14, 425-435, 1999 (see especially Table 1) which is herein incorporated in its entirety by reference.

The ability of a given aureolysin inhibitor to inhibit other enzymes e.g. MMPs can also be determined by standard assays employing the purified enzyme.

Aureolysin inhibitors include, or are expected to include, the following compounds: ilomastat (compound 1; see U.S. Pat. No. 5,183,900), marimastat (compound 3, see WO94/02447), compound 5 (see WO95/19957), solimastat (compound 7, see EP1030842), compound 9 (see WO95/19956), compound 11 (Ro 31-9790, see EP0664284, and Whittaker et al, Chemical Reviews, 99, 2735-2776, 1999) and their diastereoisomers (compound 2, compound 4, compound 6, compound 8, compound 10 and compound 12), compound 13 (Calbiochem) and stereoisomers thereof, compound 14 (Calbiochem) and phosphoramidon (compound 15) (see Table 1 below). Further examples include compounds 16 and 17.

TABLE 1
Compound 1

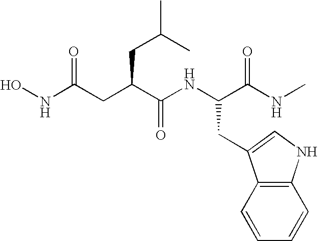

Compound 2

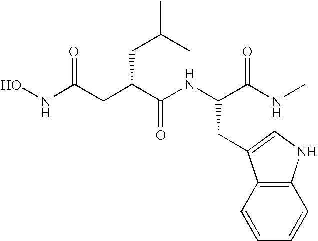

Compound 3

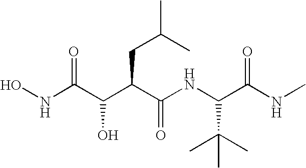

Compound 4

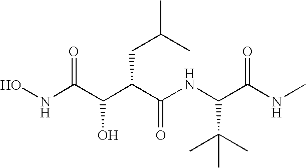

Compound 5

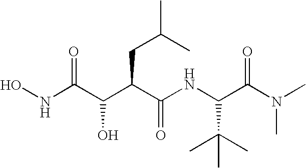

Compound 6

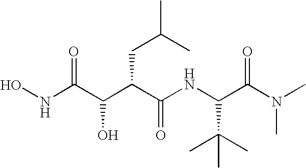

Compound 7

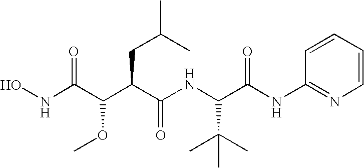

Compound 8

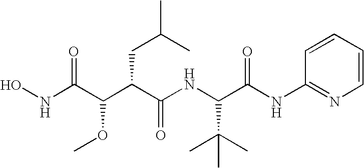

Compound 9

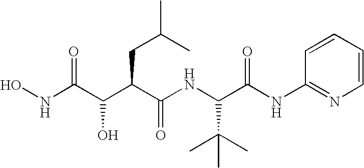

Compound 10

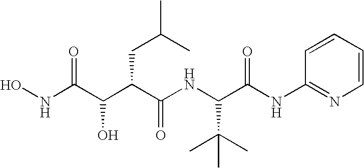

Compound 11

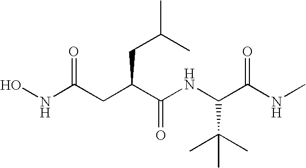

Compound 12

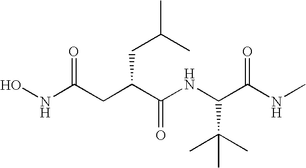

Compound 13

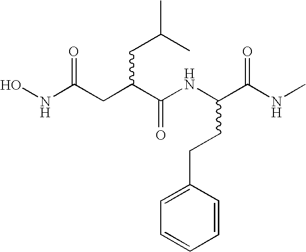

Compound 14

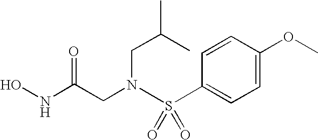

Compound 15

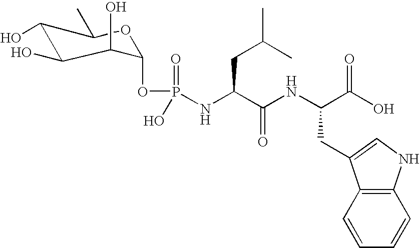

Compound 16

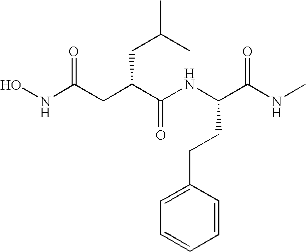

Compound 17

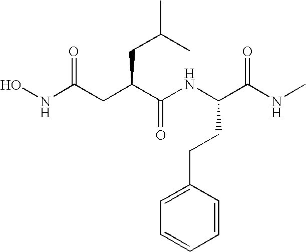

Compounds 11, 12, 16 and 17 are novel and are claimed per se, together with pharmaceutically acceptable salts and solvates thereof, as an aspect of the invention. Said compounds are claimed as solids in either amorphous or crystalline form, including all polymorphic forms. Crystalline forms may be prepared by recrystallization of the compounds from appropriate solvents. Amorphous forms may be prepared eg by spray drying a solution of the compounds.

These compounds may, for example, be prepared as described in the Examples.

As can be seen from data contained within the Examples, compound 12 is particularly interesting since it has a very balanced inhibition against aureolysin as well as against MMPs (especially MMPs 1, 2, 8 and 9). That means that when administered at a level which should inhibit aureolysin, its effect on those other MMPs would be expected to be similar. This would be expected to provide an advantage in terms of reduced tendonitis, a systemic toxicological effect common to many compounds having significant M10 inhibitory activity (e.g., compounds 3, 7, & 11).

As can also be seen from data contained within the Examples, compound 16 is particularly interesting since it has remarkably potent aureolysin inhibitory activity. In fact it was much more potent as an aureolysin inhibitor than any of the other compounds tested.

As well as claiming these novel compounds per se, we also claim processes for their preparation, their use as pharmaceuticals, pharmaceutical compositions containing them together with a pharmaceutically acceptable (particularly, a topically acceptable) diluent or carrier as well as methods of treatment of inflammatory skin diseases employing them and their use in the manufacture of a medicament for the treatment of inflammatory skin diseases.

It is preferred that the inhibitor is formulated for topical administration and it may be administered to a patient in an amount such that from 0.00001 to 10 g, preferably from 0.0001 to 1 g active ingredient is delivered per m² of the area being treated.

Typically, the total amount of inhibitor is from 0.001 to 12 wt % eg from 0.0018 to 11.6 wt %, suitably from 0.0088 to 1.4 wt %, e.g. 0.01-1.0 wt %, more suitably from 0.05 to 0.2 wt %, for example about 0.1 wt %, based on the total weight of the formulation.

The topical formulation may, for example, take the form of a gel, ointment, cream or lotion. Other example presentations include impregnated dressings, pastes, dusting powders, sprays, oils, transdermal devices etc.

The topical formulation will preferably maximise surface exposure and minimise systemic exposure to the active ingredient(s).

When said formulation is a gel it typically comprises a hydrophilic polymer such as cross-linked polyethylene glycol, cross-linked starch or polyvinyl pyrrolidone.

An ointment, cream or lotion typically contains an aqueous phase and an oleaginous phase in admixture. They may generally be characterised as oil-in-water emulsions or water-in-oil emulsions.

The formulation may additionally contain one or more emollients, emulsifiers, thickeners and/or preservatives, particularly when it is a cream or ointment.

Emollients suitable for inclusion in creams or ointments are typically long chain alcohols, for example a C8-C22 alcohol such as cetyl alcohol, stearyl alcohol and cetearyl alcohol, hydrocarbons such as petrolatum and light mineral oil, or acetylated lanolin. The total amount of emollient in the formulation is preferably about 5 wt % to about 30 wt %, and more preferably about 5 wt % to about 10 wt % based on the total weight of the formulation.

The emulsifier is typically a nonionic surface active agent, e.g., polysorbate 60 (available from ICI Americas), sorbitan monostearate, polyglyceryl-4 oleate and polyoxyethylene(4)lauryl ether. Generally the total amount of emulsifier is about 2 wt % to about 14 wt %, and more preferably about 2 wt % to about 6 wt % by weight based on the total weight of the formulation.

Pharmaceutically acceptable thickeners, such as Veegum™K (available from R. T. Vanderbilt Company, Inc.), and long chain alcohols (i.e. C8-C22 alcohols such as cetyl alcohol, stearyl alcohol and cetearyl alcohol) can be used. The total amount of thickener present is preferably about 3 wt % to about 12 wt % based on the total weight of the formulation.

Preservatives such as methylparaben, propylparaben and benzyl alcohol can be present in the formulation. Other example preservatives are phenoxyethanol and chlorocresol. The appropriate amount of such preservative(s) is known to those skilled in the art.

Optionally, an additional solubilizing agent such as benzyl alcohol, lactic acid, acetic acid, stearic acid or hydrochloric acid can be included in the formulation. If an additional solubilizing agent is used, the amount present is preferably about 1 wt % to about 12 wt % based on the total weight of the formulation.

Optionally, the formulation can contain a humectant such as glycerin and a skin penetration enhancer such as butyl stearate, urea and DMSO.

It is known to those skilled in the art that a single ingredient can perform more than one function in a cream, i.e., cetyl alcohol can serve both as an emollient and as a thickener.

Preferably, said formulation or medicament is a cream. The cream typically consists of an oil phase and a water phase mixed together to form an emulsion. Preferably, the cream comprises an oil-in-water emulsion. Preferably, the amount of water present in a cream of the invention is about 45 wt % to about 85 wt % based on the total weight of the cream.

Where the formulation or medicament is an ointment, it typically comprises a pharmaceutically acceptable ointment base such as petrolatum, or polyethylene glycol 400 (available from Union Carbide) in combination with polyethylene glycol 3350 (available from Union Carbide). The amount of ointment base present in an ointment of the invention is preferably about 60 wt % to about 95 wt % based on the total weight of the ointment.

One exemplary formulation is a cream which comprises an emulsifying ointment (e.g. around 30 wt %) comprising white soft paraffin, emulsifying wax and liquid paraffin made to 100% with purified water and containing preservative (e.g. phenoxyethanol). This formulation may also be buffered to the required pH (e.g. with citric acid and sodium phosphate). The concentration of active may typically be between 0.01 and 1.0 wt %.

In a preferred embodiment, the formulation is a cream which comprises an oil-in-water cream base comprising isostearic acid, cetyl alcohol, stearyl alcohol, white petrolatum, polysorbate 60, sorbiton monostearate, glycerin, xanthum gum, purified water, benzyl alcohol, methylparaban and propyl-paraban. Such a cream may be in the form of Aldara imiquimod cream which contains 5% imiquimod.

Compound 12 has been found to be particularly soluble in water, particularly when the solid is in amorphous form. It formulates well in oil-in-water or water-in-oil emulsions since it may be taken up in the water phase before emulsifying with the oil phase (eg paraffin).

The aureolysin inhibitor may be administered in conjunction with further medicaments, such as conventional therapies for the treatment or prevention of inflammatory skin conditions, for example antibiotics, steroids (such as hydrocortisone, clobetasone butyrate, betamethasone valerate, hydrocortisone butyrate, clobetasol propionate, fluticasone propionate, mometasone furoate and dexamethasone), non-steroidal anti-inflammatory drugs, macrolide immunosuppressants (such as cyclosporine A, tacrolimus and pimecrolimus), leukotriene antagonists and phosphodiesterase inhibitors.

These further treatments may be administered by any convenient route. Topical and oral routes are preferred.

Active agents may, where appropriate, be administered in the form of pharmaceutically acceptable salts, or solvates e.g. hydrates.

Accordingly, there is also provided a method for the treatment or prevention of an inflammatory skin condition which is characterised by colonisation with Staphylococcus aureus, comprising the topical administration of an aureolysin inhibitor in combination with administration of a further medicament.

In a further aspect of the present invention there is provided the use of an aureolysin inhibitor in the manufacture of a topical medicament for the treatment or prevention an inflammatory skin condition which is characterised by colonisation with Staphylococcus aureus in combination with a further medicament.

Combination treatments may be administered simultaneously, sequentially or separately, by the same or by different routes. In one example embodiment the further medicament may be administered orally. In another example embodiment the further medicament may be administered topically e.g. in a combined preparation with the aureolysin inhibitor.

For example the further medicament may be an antibiotic substance which is bacteriocidal for Staphylococcus aureus and which is administered orally or topically.

The appreciation of the role of aureolysin in AD allows for a novel screening method for the identification of novel substances which are aureolysin inhibitors for the treatment of AD.

Thus in another aspect of the present invention there is provided an in vitro method of screening for an agent of use in the treatment or prevention of an inflammatory skin condition which is characterised by colonisation with Staphylococcus aureus, comprising:
(i) contacting said agent with aureolysin;
(ii) determining if the aureolysin is inhibited.

Inhibition of aureolysin may be determined by a standard test, for example by means of the aureolysin inhibition assay or else by means of the milk agar plate assay described in the Examples.

There is also provided a method of screening for an agent of use in the treatment or prevention of an inflammatory skin condition which is characterised by colonisation with Staphylococcus aureus, comprising:
(i) obtaining skin washings from patients
(ii) contacting said agent with skin washings
(iii) determining (e.g. by zymography or fluorogenic enzyme assay) whether proteolytic activity is inhibited. This activity may be due to aureolysin and optionally endogenous metzincin metalloproteases.

By “agent” is meant any chemical substance, whether a “small molecule” (e.g. a molecule having a molecular weight of less than 1000 Da especially less than 600 Da), peptide, protein or antibody. Small molecules (e.g. those having a molecular weight of less than 600 Da) are preferred. Small peptides (e.g. containing less than 16 amino acid residues) are preferred. These peptides may be linear or cyclised.

In one suitable method of performing the methods and uses according to the invention, the presence of metalloprotease activity in the skin lesions associated with AD or other inflammatory condition is verified before treatment. Thus according to this aspect of the invention there is provided a method for the treatment of a skin lesion associated with an inflammatory skin condition in a mammal which is characterised by colonisation with Staphylococcus aureus which comprises (i) determining the presence of metalloprotease activity in skin washings from the locus of said skin lesion and if the presence of metalloprotease activity is confirmed then (ii) topically administering an aureolysin inhibitor to said skin lesion.

There is also provided the use of an aureolysin inhibitor in the manufacture of a topical medicament for the treatment of a skin lesion associated with an inflammatory skin condition in a mammal which is characterised by colonisation with Staphylococcus aureus, wherein said skin lesion has been pre-determined to contain metalloprotease activity.

In certain embodiments the aureolysin inhibitor is also an endogenous metzincin metalloprotease inhibitor.

In another suitable method of performing the methods and uses according to the invention, the presence of S aureus in the skin lesions associated with AD or other inflammatory condition is verified before treatment. Thus according to this aspect of the invention there is provided a method for the treatment of a skin lesion associated with an inflammatory skin condition in a mammal which is characterised by colonisation with Staphylococcus aureus which comprises (i) determining the presence of Staphylococcus aureus in the locus of said skin lesion and if the presence of Staphylococcus aureus is confirmed then (ii) topically administering an aureolysin inhibitor to said skin lesion.

There is also provided the use of an aureolysin inhibitor in the manufacture of a topical medicament for the treatment of a skin lesion associated with an inflammatory skin condition in a mammal which is characterised by colonisation with Staphylococcus aureus, wherein said skin lesion has been pre-determined to contain Staphylococcus aureus.

In certain embodiments the aureolysin inhibitor is also an endogenous metzincin metalloprotease inhibitor.

In effect the method and use according to a first aspect of the invention is preceded by a method step involving confirming the presence of Staphylococcus aureus in the locus of said skin lesion.

By “the locus of said skin lesion” is meant in and within the skin lesion or in the surrounding area.

The presence of Staphylococcus aureus may be determined directly by sampling the skin of patients and determining the presence of Staphylococcus aureus through microbiological or genetic methods. In the simplest form of assay, the affected skin is swabbed and the swab is inoculated onto blood agar plates and colonies of Staphylococcus aureus identified through standard microbiological procedures. A quantitative methodology may also be applied to assess the level of colonisation. Genetic methods such as quantitative PCR may also be used to demonstrate the presence of Staphylococcus aureus.

The presence of Staphylococcus aureus may also be determined indirectly by determining the presence of metalloprotease activity e.g. in skin washings of patients.

The presence of metalloproteases and metalloprotease activity may be detected in skin washings from patients by gelatin zymography or enzyme assay.

### EXAMPLES

### Synthetic Examples

1. Synthesis of Compounds 11 and 12

Two interchangeable routes of synthesis were used to generate compounds 11 and 12. The first was exemplified by the synthesis of compound 11 from (R)-2-(2-methoxy-2-oxoethyl)-4-methylpentanoic acid. The second was exemplified by the synthesis of compound 12 from L-leucine. The first route may be used to synthesise compound 12 by using (S)-2-(2-methoxy-2-oxoethyl)-4-methylpentanoic acid and the second may be used to synthesise compound 11 by using D-leucine in place of L-leucine.

### A. Synthesis of (R)—N1-((S)-3,3-dimethyl-1-(methylamino)-1-oxobutan-2-yl)-N4-hydroxy-2-isobutylsuccinamide (Compound 11)

i) A mixture of (R)-2-(2-methoxy-2-oxoethyl)-4-methylpentanoic acid (0.5 g, 2.4 mmol), DCC (1.2 eq, 0.61 g) and HOBT (1.02 eq, 0.34 g) in dichloromethane (5 ml) was stirred at room temperature for 10 min. (S)-2-amino-N,3,3-trimethylbutanamide (1.1 eq, 0.39 g) was added and the mixture stirred at room temperature overnight. The resulting white precipitate was filtered and the filtrate concentrated to dryness under reduced pressure to give an oil. The crude oil was dissolved in ethyl acetate (50 ml) and extracted sequentially with 2N hydrochloric acid (2×50 ml), saturated sodium bicarbonate (2×50 ml) and brine (50 ml), dried with MgSO₄ and concentrated to dryness. This residue was recrystallized from diethyl ether to give (R)-methyl 3-((S)-3,3-dimethyl-1-(methylamino)-1-oxobutan-2-ylcarbamoyl)-5-methylhexanoate as a white solid (0.60 g, 74%). A second reaction from 1.05 g of (R)-2-(2-methoxy-2-oxoethyl)-4-methylpentanoic acid gave 1.06 g (65%) of product.

¹H NMR (400 MHz, CDCl₃): 0.83 (d, 3H), 0.87 (d, 3H), 0.96 (s, 9H), 1.22 (m, 1H), 1.51 (m, 2H), 2.40 (d, 1H, j=14.5 Hz), 2.65 (m, 2H), 2.77 (s, 3H), 3.63 (s, 3H), 4.22 (d, 1H, j=9.15), 6.15 (d, 1H, j=4.39), 6.40 (d, 1H, j=9.52).

ii) (R)-methyl 3-((S)-3,3-dimethyl-1-(methylamino)-1-oxobutan-2-ylcarbamoyl)-5-methylhexanoate was converted to compound 12 using the method of Levy et al (J. Med Chem 41:199-223, 1998). Hydroxylamine hydrochloride (7.2 eq, 0.91 g) was dissolved in methanol (8.8 ml) and cooled to 0° C. Potassium hydroxide (11.4 eq, 1.17 g) in methanol (5.8 ml) was added and the mixture stirred at 0° C. for 1 h. The mixture was filtered and the filtrate added to a solution of (R)-methyl 3-((S)-3,3-dimethyl-1-(methylamino)-1-oxobutan-2-ylcarbamoyl)-5-methylhexanoate in methanol (0.6 g, 3.6 ml) and the mixture stirred at room temperature for 30 min. The reaction mixture was concentrated to dryness under reduced pressure and dissolved in water (7 ml), acidified to pH 5 with 6N hydrochloric acid and then neutralized with saturated sodium bicarbonate to pH 7. The resulting precipitate was collected by filtration and purified by flash chromatography eluting with 5% methanol/dichloromethane to 10% methanol/dichloromethane. Fractions containing product were combined and concentrated to dryness under reduced pressure. The solid was triturated in isopropyl alcohol:ethyl acetate (1:1) and filtered to give (R)—N¹—((S)-3,3-dimethyl-1-(methylamino)-1-oxobutan-2-yl)-N⁴-hydroxy-2-isobutylsuccinamide as a white solid (68 mg, 7%). A second reaction using 1.06 g of (R)-methyl 3-((S)-3,3-dimethyl-1-(methylamino)-1-oxobutan-2-ylcarbamoyl)-5-methylhexanoate in which the concentrated reaction mixture was purified directly by flash chromatography gave 0.25 g (24%) of product.

¹H NMR (400 MHz, DMSO): 0.76 (d, 3H), 0.80 (d, 3H), 0.86 (s, 9H), 1.05 (m, 1H), 1.40 (m, 2H), 2.09 (m, 2H), 2.54 (s, 3H), 2.82 (m, 1H), 4.13 (m, 1H, j=9.5 Hz), 7.64 (d, 1H, j=9.89), 7.84 (d, 1H, j=4.76), 8.68 (br s, 1H), 10.35 (br s, 1H).

### B. Synthesis of (S)—N1-((S)-3,3-dimethyl-1-(methylamino)-1-oxobutan-2-yl)-N4-hydroxy-2-isobutylsuccinamide (Compound 12)

i) Sodium nitrite (84.2 g, 1.22 mol) was added stepwise at 0° C. to an ice-cold solution of L-leucine (100 g, 0.762 mol) in 48% aqueous HBr (836 g) and water (360 ml). When addition was complete the reaction was held at 0° C. for 1 h and then allowed to warm to room temperature overnight. The reaction mixture was then re-cooled to 0° C. and quenched by stepwise addition of sodium carbonate (˜135 g) until the reaction mixture reached pH 4.5. The mixture was then extracted into dichloromethane (2×500 ml), dried with MgSO₄ and the solvent removed in vacuo to yield (S)-2-bromo-4-methylpentanoic acid as an orange oil (115.8 g, 80% yield) that lost its colour on standing.

¹H NMR (CDCl₃): δ 0.92 (3H, d, J=6.5 Hz), 0.96 (3H, d, J=6.5 Hz), 1.80 (1H, m), 1.91 (2H, m), 4.28 (1H, t, J=7.85 Hz), 11.76 (1H, br s).

α_{D} −39.6°, c=2.118 in MeOH.

ii) Boron trifluoride dietherate (7.9 g, 0.0559 mol) was added to a solution of (S)-2-bromo-4-methylpentanoic acid (108 g, 0.559 mol) in tert-butyl acetate (450 ml) and the reaction mixture stirred overnight under nitrogen at room temperature. The mixture was poured into saturated sodium bicarbonate and the basic pH maintained by the addition of more sodium bicarbonate. The organic phase was separated, washed with brine (2×200 ml), dried over MgSO₄ and evaporated to dryness to give 135 g crude oil. The oil was then purified by vacuum distillation (55° C. at 4 mbar to give a theoretical boiling point of 190° C.). (S)-tert-butyl 2-bromo-4-methylpentanoate was collected as a colourless oil (91.7 g, 66% yield).

¹H NMR (CDCl₃): δ 0.89 (3H, d, J=6.5 Hz), 0.93 (3H, d, J=6.5 Hz), 1.46 (9H, s), 1.72 (1H, m), 1.84 (2H, m), 4.15 (1H, t, J=7.65 Hz).

α_{D} −29.91°, c=1.906 in MeOH.

iii) Potassium tert-butoxide (38.8 g, 0.346 mol) was added stepwise at 0° C. under nitrogen to a solution of dibenzyl malonate (98.3 g, 0.346 mol) in dry dimethylformamide (169 ml) until dissolved. The solution was held at 0° C. and a solution of (S)-tert-butyl 2-bromo-4-methylpentanoate (86.8 g, 0.346 mols) in dry dimethylformamide (160 ml) was added dropwise over a period of 1 h and then stirred at 0° C. for 4 days. The reaction was warmed to room temperature, diluted with ethyl acetate (600 ml) and saturated aqueous ammonium chloride solution (400 ml) added. The organic layer was separated and the aqueous layer re-extracted with ethyl acetate (400 ml). The organic layers were combined and washed with 10% sodium chloride solution (500 ml) and dried over MgSO₄. The solvent was removed to give 195 g pale yellow oil. The oil was purified by flash column chromatography (10:1 heptane:ethyl acetate) to give 63 g (40% yield) of (S)-1,1-dibenzyl 2-tert-butyl 4-methylpentane-1,1,2-tricarboxylate as a clear oil that solidified to a white solid on standing.

¹H NMR (CDCl₃): δ0.82 (6H, m), 1.37 (9H, s), 1.50 (3H, m), 3.03 (1H, dt, J¹=4.28 Hz, J²=10.32 Hz), 3.73 (1H, d, J=10.20 Hz), 5.11 (4H, m), 7.29 (10H, m).

α_{D} −15.93°, c=1.695 in MeOH.

iv) TFA (100 ml) was added to a solution of (S)-1,1-dibenzyl 2-tert-butyl 4-methylpentane-1,1,2-tricarboxylate (60 g, 0.132 mol) in dichloromethane (500 ml) and the reaction stirred at room temperature overnight. The solvent was removed under vacuum and the resulting oil re-dissolved in dichloromethane (300 ml). The material was then washed with water (300 ml), dried with MgSO₄ and the solvent removed to give a pale orange oil (51.1 g, α_{D} −7.44°, c=2.554 in MeOH). The oil was dissolved in 180 ml diethyl ether, 520 ml n-hexane added and the solution cooled in an ice bath. The resultant precipitate was filtered and the filtrate concentrated in vacuo to yield (S)-2-(1,3-bis(benzyloxy)-1,3-dioxopropan-2-yl)-4-methylpentanoic acid as a pale oil (31.06 g, 60% yield).

¹H NMR (CDCl3): δ 0.81 (3H, d, J=3.47 Hz), 0.83 (3H, d, J=3.47 Hz), 1.16 (1H, m), 1.59 (2H, m), 3.18 (1H, m), 3.79 (1H, d, J=9.79 Hz), 5.14 (4H, m), 7.31 (10H, m).

α_{D} −40.10°, c=2.319 in MeOH

v) HOBT (11.2 g, 0.083 mol) was added to a solution of (S)-2-(1,3-bis(benzyloxy)-1,3-dioxopropan-2-yl)-4-methylpentanoic acid (30 g, 0.075 mol) in ethyl acetate (200 ml) and dimethylformamide (10 ml). The reaction was cooled to 0° C. and a solution of DCC (19.2 g, 0.093 mol) in ethyl acetate (40 ml, 2 vol) added over 15 min. The reaction was allowed to warm to room temperature and stirred for 1 h. The DCU was removed by filtration, the reaction cooled again to 0° C., a solution of (S)-2-amino-N,3,3-trimethylbutanamide (10.86 g, 0.075 mol) in ethyl acetate (20 ml, 2 vol) added and the reaction stirred at room temperature for 2 days. The reaction was then washed with 2M sodium carbonate (200 ml), water (200 ml), 2M sodium carbonate (200 ml) again, brine (200 ml) and water (200 ml). The organic layer was dried with MgSO₄, filtered and concentrated in vacuo to give 35 g pale yellow waxy solid. This solid was slurried in diethyl ether and dibenzyl 2-((S)-1-((S)-3,3-dimethyl-1-(methylamino)-1-oxobutan-2-ylamino)-4-methyl-1-oxopentan-2-yl)malonate filtered off as a white solid (24.64 g, 62% yield).

¹H NMR (CDCl3): δ 0.81 (3H, d, J=3.06 Hz), 0.82 (3H, d, J=3.06 Hz), 0.98 (9H, m), 0.99 (1H, m), 1.57 (1H, m), 1.69 (1H, m), 2.67 (3H, d, J=4.90 Hz), 2.94 (1H, dt, J1=3.67 Hz, J2=10.20 Hz), 3.78 (1H, d, J=10.20 Hz), 4.13 (1H, d, J=8.77 Hz) 5.08 (4H, m), 6.22 (1H, d, J=4.49 Hz), 6.33 (1H, d, J=8.77 Hz) 7.29 (10H, m).

α_{D} −36.00°, c=1.750 in MeOH.

M. P. 98-99° C.

vi) Dibenzyl 2-((S)-1-((S)-3,3-dimethyl-1-(methylamino)-1-oxobutan-2-ylamino)-4-methyl-1-oxopentan-2-yl)malonate (24.5 g, 0.047 mol) was dissolved in ethanol (300 ml) and the solution purged with nitrogen. The flask was evacuated, purged with nitrogen again and 10% palladium on carbon catalyst (2.45 g, 10% wt) added. The flask was re-evacuated, purged with nitrogen once more and then evacuated and purged three times with hydrogen. The reaction was left under an atmosphere of hydrogen and stirred at room temperature over the weekend. The catalyst was filtered off and the solvent removed under vacuum to give 15 g 2-((S)-1-((S)-3,3-dimethyl-1-(methylamino)-1-oxobutan-2-ylamino)-4-methyl-1-oxopentan-2-yl)malonic acid as a white solid (100% yield).

¹H NMR (DMSO): δ 0.82 (3H, d, J=6.53 Hz), 0.90 (12H, m), 1.05 (1H, m), 1.51 (2H, H, m), 2.57 (3H, d, J=4.49 Hz), 3.09 (1H, dt, J¹=3.88 Hz, J²=8.32 Hz), 3.33 (1H, d, J=10.20 Hz), 4.07 (1H, d, J=9.18 Hz), 7.55 (1H, m) 8.12 (1H, d, J=9.18 Hz).

α_{D} −80.50°, c=1.913 in MeOH.

M. P. 101° C.

vii) 2-((S)-1-((S)-3,3-dimethyl-1-(methylamino)-1-oxobutan-2-ylamino)-4-methyl-1-oxopentan-2-yl)malonic acid (14.5 g) was dissolved in ethanol, carbon (1.45 g) added and the reaction stirred overnight at 80° C. The carbon was filtered off and the solvent removed in vacuo to give 11.51 g of (S)-3-((S)-3,3-dimethyl-1-(methylamino)-1-oxobutan-2-ylcarbamoyl)-5-methylhexanoic acid as a pale grey solid.

¹H NMR (DMSO): δ 0.81 (3H, d, J=6.32 Hz), 0.87 (12H, m), 1.09 (1H, m), 1.45 (2H, m), 2.19 (1H, dd, J¹=6.53 Hz, J²=16.12 Hz), 2.36 (1H, dd, J¹=7.75 Hz, J²=16.12 Hz), 2.54 (3H, d, J=4.49 Hz), 2.91 (1H, m), 4.12 (1H, d, J=9.18 Hz), 7.78 (1H, m) 7.95 (1H, d, J=9.18 Hz).

α_{D} −42.21°, c=1.919 in MeOH.

M. P. 204-205° C.

viii) O-benzylhydroxylamine hydrochloride (9.30 g, 0.058 mol), NMM (5.93 g, 0.059 mol), HOBT (6.42 g, 0.048 mol) and EDAC (9.11 g, 0.048 mol) were added to a stirred solution of (S)-3-((S)-3,3-dimethyl-1-(methylamino)-1-oxobutan-2-ylcarbamoyl)-5-methylhexanoic acid (11.51 g, 0.038 mol) in dimethylformamide (161 ml) and dichloromethane (205 ml) at 0° C. The reaction mixture was left to warm to room temperature and stirred overnight. It was then diluted with dichloromethane (500 ml) and washed sequentially with water (500 ml), 0.6N HCl (500 ml), saturated sodium carbonate (500 ml) and water (4×500 ml). The organic layer was dried and the solvent removed in vacuo to give (S)—N⁴-(benzyloxy)-N¹—((S)-3,3-dimethyl-1-(methylamino)-1-oxobutan-2-yl)-2-isobutylsuccinamide as a white solid (7.35 g, 43% yield).

¹H NMR (DMSO): δ 0.81 (3H, d, J=6.32 Hz), 0.87 (3H, d, J=6.53 Hz), 0.90 (9H, m), 1.00 (1H, m), 1.42 (2H, m), 1.97 (1H, dd, J¹=7.34 Hz, J²=14.4 Hz), 2.11 (1H, dd, J¹=7.14 Hz, J²=15.44 Hz), 2.52 (3H, d, J=4.49 Hz), 2.96 (1H, m), 4.12 (1H, d, J=9.38 Hz), 4.73 (2H, q, J¹=11.02 Hz, J²=9.7 Hz), 7.38 (5H, m), 7.85 (1H, m), 7.97 (1H, d, J=9.18 Hz).

α_{D} −19.37°, c=1.497 in MeOH.

M. P. 128-129° C.

ix) (S)—N⁴-(benzyloxy)-N¹—((S)-3,3-dimethyl-1-(methylamino)-1-oxobutan-2-yl)-2-isobutylsuccinamide (7.35 g) was dissolved in ethanol (100 ml) and the solution purged with nitrogen. The flask was evacuated, purged with nitrogen again and 10% palladium on carbon catalyst (735 mg, 10% wt) added. The flask was re-evacuated, purged with nitrogen once more and then evacuated and purged three times with hydrogen. The reaction mixture was then left under an atmosphere of hydrogen and stirred at room temperature over the weekend. The catalyst was filtered off and the solvent removed under vacuum to give 5.12 g of (S)—N¹—((S)-3,3-dimethyl-1-(methylamino)-1-oxobutan-2-yl)-N⁴-hydroxy-2-isobutylsuccinamide (compound 12) as a white solid (98% yield).

¹H NMR (D4-MeOH): δ 0.89 (3H, d, J=6.53 Hz), 0.94 (3H, d, J=6.32 Hz), 1.01 (9H, s), 1.17 (1H, m), 1.57 (2H, m), 2.14 (1H, dd, J¹=6.12 Hz, J²=14.68 Hz), 2.33 (1H, dd, J¹=8.36 Hz, J²=14.68 Hz), 2.71 (3H, s), 2.93 (1H, m), 4.10 (1H, s).).

α_{D} −33.1°, c=1.60 in MeOH.

2. Synthesis of Compounds 16 and 17

(S)-2-amino-N-methyl-4-phenylbutanamide was prepared as follows:

i) To a stirred solution of (S)-2-amino-4-phenylbutanoic acid (5.0 g, 27.9 mmol) in methanol (25 ml) was added thionyl chloride (2.26 ml, 30.69 mmol) at 0° C. The mixture was warmed up to room temperature then heated at 65° C. for 2 h. After concentration in vacuo the residue was triturated with diethyl ether (10 ml) and the solid collected by suction filtration, washed with diethyl ether (5 ml) and air dried to give (S)-methyl 2-amino-4-phenylbutanoate as its hydrochloride salt (6.11 g, 95%).

LCMS (3 min) purity=97%, tᵣ=1.05, m/z 194 [M+H]⁺.

ii) To a solution of 8M methylamine in ethanol (8.7 ml, 69.6 mmol) was added (S)-methyl 2-amino-4-phenylbutanoate hydrochloride (4.0 g, 17.4 mmol) at room temperature. Stirring was continued overnight. The reaction mixture was concentrated in vacuo, diethyl ether (5 ml×3) added and evaporation repeated. The solid was suspended in dichloromethane (30 ml), washed with saturated aqueous sodium bicarbonate (10 ml) and water (10 ml), dried (Na₂SO₄), filtered and concentrated in vacuo to give (S)-2-amino-N-methyl-4-phenylbutanamide as a white solid (2.77 g, 83%).

LCMS (3 min) purity=94%, tᵣ 1.05, m/z 193 [M+H], ¹H NMR (MeOD) δ 1.65 (1H, m), 1.85 (1H, m), 2.55 (2H, m), 2.60 (3H, s, CONMe), 3.15 (1H, m), 7.00-7.20 (5H, m, Ar).

This material was used in the synthesis of both compounds 16 and 17 as described below.

### A. Synthesis of (S)—N4-hydroxy-2-isobutyl-N1-((S)-1-(methylamino)-1-oxo-4-phenylbutan-2-yl)succinamide (Compound 16)

Two routes of synthesis to compound 16 designated route A and route B were used.

Route A to Compound 16

i) To a mixture of (S)-3-(methoxycarbonyl)-5-methylhexanoic acid (250 mg, 1.33 mmol), EDC (331 mg, 1.726 mmol) and HOBT (233 mg, 1.726 mmol) in THF (5 ml) was added (S)-2-amino-N-methyl-4-phenylbutanamide (281 mg, 1.46 mmol) followed by triethylamine (0.46 ml, 3.30 mmol). The reaction mixture was stirred at room temperature overnight. After removal of volatiles the residue was taken up into ethyl acetate (10 ml), washed with 10% citric acid (5 ml), followed by saturated aqueous sodium bicarbonate (5 ml) and water (5 ml). The ethyl acetate layer was dried (Na₂SO₄), filtered and concentrated in vacuo to give (S)-methyl 4-methyl-2-(2-((S)-1-(methylamino)-1-oxo-4-phenylbutan-2-ylamino)-2-oxoethyl)pentanoate (380 mg, 79%).

LCMS (3 min) purity=93%, tᵣ 1.97, m/z 363 [M+H]⁺, 725 [M+H]⁺.

¹H NMR (MeOD) δ1.80 (6H, m, isobutyl), 1.20 (1H, m, isobutyl), 1.55 (2H, m, isobutyl), 1.80 (1H, m), 2.05 (1H, m), 2.30 (1H, dd, J=14.91 and 5.62 Hz), 2.40 (1H, dd, J=14.91 and 9.29 Hz), 2.45 (2H, m), 2.60 (3H, s, CONHMe), 2.85 (1H, m), 3.55 (3H, s, CO₂Me), 4.15 (1H, dd, J=9.54 and 4.89 Hz), 7.00-7.20 (5H, m, Ar).

ii) To a solution of (S)-methyl 4-methyl-2-(2-((S)-1-(methylamino)-1-oxo-4-phenylbutan-2-ylamino)-2-oxoethyl)pentanoate (250 mg, 0.69 mmol) in a mixture of THF (2.0 ml) and methanol (2.0 ml) was added 2M aqueous sodium hydroxide (1.0 ml) at room temperature. After stirring at room temperature for 1 h the reaction mixture was concentrated to about 0.5 ml and acidified carefully with 1M aqueous hydrochloric acid. The aqueous layer was extracted with ethyl acetate (2×3 ml) and the combined organic layers dried (Na₂SO₄), filtered and concentrated in vacuo to give a mixture of (S)-5-methyl-3-((S)-1-(methylamino)-1-oxo-4-phenylbutan-2-ylcarbamoyl)hexanoic acid (isomer A) and (S)-4-methyl-2-(2-((S)-1-(methylamino)-1-oxo-4-phenylbutan-2-ylamino)-2-oxoethyl)pentanoic acid (isomer B) (210 mg, 87%); this was used directly in step (iii).

LCMS (3 min) purity=72% (isomer A, tᵣ=1.69)+10% (isomer B, tᵣ=1.67), m/z 349 [M+H]⁺.

iii) To the mixture of carboxylic acids obtained from step (ii) (210 mg, 0.603 mmol), EDC (150 mg, 0.784 mmol) and HOBT (105 mg, 0.784 mmol) in THF (5 ml) was added followed by O-tetrahydro-2H-pyran-2-yl-hydroxylamine (92 mg, 0.784 mmol) and triethylamine (0.20 ml, 1.50 mmol). The reaction mixture was stirred at room temperature overnight. After removal of volatiles the residue was taken up in ethyl acetate (10 ml), washed with 10% citric acid (5 ml), followed by saturated aqueous sodium bicarbonate (5 ml) and water (5 ml). The ethyl acetate layer was dried (Na₂SO₄), filtered and concentrated in vacuo to give (2S)-2-isobutyl-N¹—((S)-1-(methylamino)-1-oxo-4-phenylbutan-2-yl)-N⁴-(tetrahydro-2H-pyran-2-yloxy)succinamide (isomer A) and (2S)-2-isobutyl-N⁴—((S)-1-(methylamino)-1-oxo-4-phenylbutan-2-yl)-N¹-(tetrahydro-2H-pyran-2-yloxy)succinamide (isomer B) (240 mg, 89%).

LCMS (3 min) purity=61%, (isomer A, tᵣ=1.77)+13% (isomer B, tᵣ=1.74), m/z 448 [M+H]⁺, 364 [M+H−THP]⁺.

iv) To a solution of (2S)-2-isobutyl-N¹—((S)-1-(methylamino)-1-oxo-4-phenylbutan-2-yl)-N⁴-(tetrahydro-2H-pyran-2-yloxy)succinamide and (2S)-2-isobutyl-N⁴—((S)-1-(methylamino)-1-oxo-4-phenylbutan-2-yl)-N¹-(tetrahydro-2H-pyran-2-yloxy)succinamide (240 mg, 0.537 mmol) in methanol (7 ml) was added Amberlyst H-15 resin (200 mg). The mixture was agitated at room temperature for 3 h, filtered and concentrated in vacuo. The desired compound was then isolated by preparative reverse phase HPLC. Crude product in 2:1 dimethyl sulphoxide:acetonitrile (1.6 ml) was injected onto a ThermoHypersil-Keystone Hyperprep HS C18 column (12 μm, 100×21.2 mm) and eluted over 9.5 min (30 ml/min) with a 20-100% gradient of acetonitrile/0.1% TFA (solvent B) in water/0.1% TFA (solvent A) using UV detection at 215 nm to yield compound 16 as a white solid (60 mg, 32%).

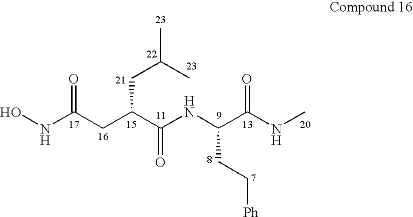

LCMS (7 min) purity=100% (tᵣ=3.48), m/z 364 [M+H]⁺. ¹H NMR (MeOD) δ 0.80 (3H, d, J=6.36 Hz, H-23), 1.90 (3H, d, J=6.36 Hz, H-23), 1.15 (1H, m, H-21), 1.45 (2H, m, H-21 and H-22), 1.75 (1H, m, H-8), 2.10 (1H, dd, J=15.16 and 4.15 Hz, H-16), 2.15 (1H, m, H-8), 2.35 (1H, dd, J=10.5 and 15.16 Hz, H-16), 2.50 (1H, m, H-7), 2.65 (1H, m, H-7), 2.65 (3H, s, H-20), 2.70 (1H, m, H-15), 4.15 (1H, dd, J=11, 3.66, H-9 Hz), 7.05-7.20 (5H, m, Ar). ¹³C NMR (MeOD) δ23.29, 24.74, 27.52, 28.04, 34.43, 35.19, 37.70, 43.20, 44.18, 55.53, 128.18, 130.50, 130.52, 143.19, 172.13 (C-17), 175.93 (C-13), 179.17 (C-11).

Route B to Compound 16

i) To a stirring mixture of (S)-3-(methoxycarbonyl)-5-methylhexanoic acid (100 mg, 0.532 mmol) and Boc₂O (139 mg, 0.638 mmol) in t-BuOH (99%, 1.5 ml) was added DMAP (19.5 mg, 0.159 mmol) at room temperature. After stirring for 1.5 h the reaction mixture was concentrated in vacuo, diluted with ethyl acetate (5 ml), washed with saturated aqueous citric acid (2×2 ml), followed by saturate aqueous sodium bicarbonate (2×2 ml) and water (2 ml). The ethyl acetate layer was dried (Na₂SO₄), filtered and concentrated in vacuo to give (S)-4-tert-butyl 1-methyl 2-isobutylsuccinate as a yellow viscous oil (100 mg). This compound was used in the next step without further purification.

¹H NMR (CDCl₃) δ0.75 (6H, m, i-butyl), 1.15 (1H, m, i-butyl), 1.20 (9H, s, t-butyl), 1.35 (2H, m, i-butyl), 2.15 (1H, dd, J=16.28 and 5.21 Hz), 2.35 (1H, dd, J=16.28, 9.33 Hz), 2.65 (1H, m), 3.40 (3H, s, CO₂Me).

ii) To a solution of (S)-4-tert-butyl 1-methyl 2-isobutylsuccinate (100 mg, 0.41 mmol) in methanol (0.8 ml) was added potassium carbonate (68 mg, 0.492 mmol) and water (0.2 ml). The mixture was heated at 55° C. for 18 h. The reaction mixture was then concentrated, redissolved in ethyl acetate (5 ml) and carefully acidified with 1M aqueous hydrochloric acid. The aqueous layer was extracted with ethyl acetate (2 ml) and the combined ethyl acetate layers, dried (Na₂SO₄), filtered and concentrated in vacuo to give (S)-2-(2-tert-butoxy-2-oxoethyl)-4-methylpentanoic acid as a crude product (60 mg) that was used directly in step (iii).

iii) To a mixture of crude (S)-2-(2-tert-butoxy-2-oxoethyl)-4-methylpentanoic acid (90 mg, 0.39 mmol), EDC (97 mg, 0.507 mmol) and HOBT (68 mg, 0.507 mmol) in DMF (1 ml) was added (S)-2-amino-N-methyl-4-phenylbutanamide (90 mg, 0.468 mmol) followed by triethylamine (0.135 ml, 0.97 mmol). The reaction mixture was stirred at room temperature overnight. After removal of volatiles the residue was taken up in ethyl acetate (5 ml), washed with 10% citric acid (2 ml), followed by saturated aqueous sodium bicarbonate (2 ml) and water (2 ml). The ethyl acetate layer was dried (Na₂SO₄), filtered and concentrated in vacuo to give (S)-tert-butyl 5-methyl-3-((S)-1-(methylamino)-1-oxo-4-phenylbutan-2-ylcarbamoyl)hexanoate (65 mg, 79%); this was used directly in step (iv).

LCMS (3 min) purity=47%, tᵣ=2.16, m/z 405 [M+H]⁺.

iv) To a solution of (S)-tert-butyl 5-methyl-3-((S)-1-(methylamino)-1-oxo-4-phenylbutan-2-ylcarbamoyl)hexanoate (65 mg, 0.161 mmol) in dichloromethane (0.6 ml) was added TFA (0.24 ml) at room temperature. After standing for 45 min the reaction mixture was evaporated to dryness. Dichloromethane (2×0.5 ml) was added and evaporation repeated to give (S)-5-methyl-3-((S)-1-(methylamino)-1-oxo-4-phenylbutan-2-ylcarbamoyl)hexanoic acid as a yellow viscous oil (58 mg, quantitative).

LCMS (3 min) purity=49%, tᵣ=1.69, m/z 349 [M+H]⁺.

v) To a mixture of (S)-5-methyl-3-((S)-1-(methylamino)-1-oxo-4-phenylbutan-2-ylcarbamoyl)hexanoic acid (58 mg, 0.167 mmol), EDC (66 mg, 0.344 mmol) and HOBT (46 mg, 0.344 mg) in THF (2 ml) was added O-tetrahydro-2H-pyran-2-yl-hydroxylamine (40.3 mg, 0.784 mmol) and triethylamine (0.084 ml, 0.60 mmol). After stirring at room temperature for 18 h, the reaction mixture was evaporated, redissolved in ethyl acetate (2 ml), washed with 10% citric acid (0.5 ml), followed by saturated aqueous sodium bicarbonate (0.5 ml) and water (0.5 ml). The ethyl acetate layer was dried (Na₂SO₄), filtered and concentrated in vacuo to give crude (2S)-2-isobutyl-N¹—((S)-1-(methylamino)-1-oxo-4-phenylbutan-2-yl)-N⁴-(tetrahydro-2H-pyran-2-yloxy)succinamide (50 mg, 67%).

LCMS (3 min) purity=44%, tᵣ=1.77, m/z 448 [M+H]⁺, 364 [M+H−THP]⁺.

vi) (2S)-2-isobutyl-N¹—((S)-1-(methylamino)-1-oxo-4-phenylbutan-2-yl)-N⁴-(tetrahydro-2H-pyran-2-yloxy)succinamide (50 mg, 0.11 mmol) was deprotected to give compound 16 using the procedure described under Route A. Yield=1.8 mg, 4.5% (over 6 steps).

LCMS (7 min) purity=100% (tᵣ=3.48), m/z 364 [M+H]. ¹H NMR was identical to that prepared via Route A.

### B. Synthesis of (R)—N4-hydroxy-2-isobutyl-N1-((S)-1-(methylamino)-1-oxo-4-phenylbutan-2-yl)succinamide (Compound 17)

i) To a mixture of (R)-3-(methoxycarbonyl)-5-methylhexanoic acid (250 mg, 1.32 mmol), EDC (332 mg, 1.73 mmol) and HOBT (234 mg, 1.73 mmol) in THF (5 ml) was added O-tetrahydro-2H-pyran-2-yl-hydroxylamine (202 mg, 1.73 mmol) and Et₃N (0.46 ml, 3.30 mmol). The reaction mixture was stirred at room temperature overnight. After removal of the volatiles the residue was taken up in ethyl acetate (15 ml), washed with 10% citric acid (5 ml), followed by saturated aqueous sodium bicarbonate (5 ml) and water (5 ml). The ethyl acetate layer was dried (Na₂SO₄), filtered and concentrated in vacuo to give (2R)-methyl 4-methyl-2-(2-oxo-2-(tetrahydro-2H-pyran-2-yloxyamino)ethyl)pentanoate as a colourless viscous oil (0.33 g, 85%).

LCMS (3 min) purity=67%, tᵣ=1.84, m/z 288 [M+H]⁺, 204 [M+H−THP]⁺.

ii) To a solution of (2R)-methyl 4-methyl-2-(2-oxo-2-(tetrahydro-2H-pyran-2-yloxyamino)ethyl)pentanoate (320 mg, 1.11 mmol) in a mixture of THF (3.0 ml) and methanol (1.5 ml) was added 2M aqueous sodium hydroxide (1.5 ml) at room temperature. After stirring at room temperature for 1 h the reaction mixture was concentrated to about 0.5 ml and acidified carefully with 1M aqueous hydrochloric acid. The aqueous layer was extracted with ethyl acetate (2×5 ml) and the combined organic layers dried (Na₂SO₄), filtered and concentrated in vacuo to give a mixture of (2R)-4-methyl-2-(2-oxo-2-(tetrahydro-2H-pyran-2-yloxyamino)ethyl)pentanoic acid and (3R)-5-methyl-3-(tetrahydro-2H-pyran-2-yloxycarbamoyl)hexanoic acid (260 mg, 86%) that was used directly in step (iii).

LCMS (3 min) purity=70%, tᵣ=1.59 (isomers co-elute), m/z 274 [M+H]⁺, 190 [M+H−THP]⁺.

iii) To a mixture of (2R)-4-methyl-2-(2-oxo-2-(tetrahydro-2H-pyran-2-yloxyamino)ethyl)pentanoic acid and (3R)-5-methyl-3-(tetrahydro-2H-pyran-2-yloxycarbamoyl)hexanoic acid (120 mg, 0.439 mmol), EDC (109 mg, 0.571 mmol) and HOBT (77 mg, 0.571 mmol) in DMF (3 ml) was added (S)-2-amino-N-methyl-4-phenylbutanamide (102 mg, 0.53 mmol), followed by triethylamine (0.151 ml, 1.09 mmol). The reaction mixture was stirred at room temperature overnight then diluted with ethyl acetate (10 ml), washed with 10% citric acid (5 ml), followed by saturated aqueous sodium bicarbonate (5 ml) and water (5 ml). The ethyl acetate layer was dried (Na₂SO₄), filtered and concentrated in vacuo to give a mixture of (2R)-2-isobutyl-N¹—((S)-1-(methylamino)-1-oxo-4-phenylbutan-2-yl)-N⁴-(tetrahydro-2H-pyran-2-yloxy)succinamide (isomer A), (2R)-2-isobutyl-N⁴—((S)-1-(methylamino)-1-oxo-4-phenylbutan-2-yl)-N¹-(tetrahydro-2H-pyran-2-yloxy)succinamide (isomer B) and (3R)-3-isobutyl-1-(tetrahydro-2H-pyran-2-yloxy)pyrrolidine-2,5-dione (80 mg).

LCMS (3 min) showed 23% of isomers A and B (co-eluting at tᵣ=1.77), m/z 448 [M+H]⁺, 346 [M+H−THP]⁺ and 69% of the by-product (3R)-3-isobutyl-1-(tetrahydro-2H-pyran-2-yloxy)pyrrolidine-2,5-dione (tᵣ=2.0), m/z 319 [M+Na+MeCN]⁺, 533 [M+Na]⁺. Preparative reverse phase HPLC purification of the crude product in 2:1 dimethyl sulphoxide:acetonitrile (1.6 ml) injected onto a ThermoHypersil-Keystone Hyperprep HS C18 column (12 μm, 100×21.2 mm) isolated fractions containing a mixture of isomers A and B. The column was eluted over 9.5 min at 30 ml/min with a 20-100% gradient of acetonitrile/0.1% TFA (solvent B) in water/0.1% TFA (solvent A) using UV detection at 215 nm.

iv) Fractions containing (2R)-2-isobutyl-N¹—((S)-1-(methylamino)-1-oxo-4-phenylbutan-2-yl)-N⁴-(tetrahydro-2H-pyran-2-yloxy)succinamide and (2R)-2-isobutyl-N⁴—((S)-1-(methylamino)-1-oxo-4-phenylbutan-2-yl)-N¹-(tetrahydro-2H-pyran-2-yloxy)succinamide from step (iii) were allowed to stand at room temperature overnight. Concentration in vacuo gave the deprotected products (10 mg), from which compound 17 (1.1 mg) was purified by preparative HPLC using the method described in step (iii).

LCMS (7 min) purity=86%, tᵣ=3.21, m/z 364 [M+H]⁺, 386 [M+Na]⁺, ¹H NMR (MeOD) δ 1.75 (3H, d, J=6.35 Hz, isobutyl), 1.85 (3H, d, J=6.35 Hz, isobutyl), 1.15 (1H, m, isobutyl), 1.45 (2H, m, isobutyl), 1.90 (1H, m), 2.10 (1H, dd, J=14.45 and 5.85 Hz), 2.25 (1H, m), 2.40-2.60 (2H, m), 2.60 (3H, s, CONHMe), 2.75 (1H, m), 4.10 (1H, dd, J=9.15 and 5.21 Hz), 7.00-7.15 (5H, m, Ar).

### Biological Examples

1. Aureolysin and MMP Enzyme Inhibition Assays

Compound inhibitory activity against purified aureolysin type I and type II (BioCentrum Ltd) was assessed in mixtures (0.1 ml) containing 90 mM MOPS buffer pH 6.8, 4.5 mM calcium chloride, 0.045% Brij 35, 10 μM Mca-Pro-Leu-Gly-Leu-Dap(Dnp)-Ala-Arg-amide (Bachem), aureolysin and 2% dimethyl sulphoxide vehicle with or without inhibitor. Compound activity against human MMPs 1, 2, 8 and 9 (Calbiochem) was assessed in the same way except that the buffer used was 90 mM Tris-HCl pH 7.5@25° C., 90 mM sodium chloride, 9 mM calcium chloride and 0.045% Brij 35. Reactions were incubated at 37° C. for 1 hour, stopped with 0.1 ml 0.5M acetic acid and the fluorescence measured using 320 nm excitation and 405 nm emission. The compound concentration eliciting a 50% decrease in enzyme activity under assay conditions (the IC₅₀ value) was determined by curve fitting (XLfit, IDBS Ltd). Results are shown in Table 2.

TABLE 2

| | IC₅₀ value (μM) or % inhibition | | | | | |
|---|---|---|---|---|---|---|
| Cpd | Aureolysin | Aureolysin | | | | |
| # | type I | type II | MMP-1 | MMP-2 | MMP-8 | MMP-9 |
| 1 | 0.26 | 0.43 | 0.00092 | 0.00044 | 0.00033 | 0.00046 |
| 3 | 9.9 | 14 | 0.00076 | 0.00071 | 0.00053 | 0.0016 |
| 5 | 27 | 41 | 0.011 | 0.0077 | 0.0039 | 0.030 |
| 7 | 2.2 | 3.5 | 0.0033 | 0.0032 | 0.0017 | 0.0051 |
| 9 | 1.5 | 2.4 | 0.0031 | 0.0018 | 0.0017 | 0.0041 |
| 11 | 1.3 | 2.4 | 0.0028 | 0.0019 | 0.0014 | 0.0036 |
| 12 | 1.3 | 2.2 | 2.3 | 2.7 | 1.4 | 4.6 |
| 13 | 0.33 | 0.69 | 0.0056 | 0.0011 | 0.0016 | 0.0019 |
| 14 | 1.0 | 2.5 | 0.29 | 0.0046 | 0.021 | 0.010 |
| 15 | 2.8 | 2.3 | 0% @ | 0% @ | 0% @ | 0% @ |
| | | | 2.7 μM | 2.7 μM | 2.7 μM | 2.7 μM |
| 16 | 0.057 | 0.093 | 6.2 | 2.1 | 1.6 | 2.0 |
| 17 | 1.2 | 2.3 | 0.017 | 0.0024 | 0.0044 | 0.0059 |

2. Protease Activity in the Skin Washings of Eczema Patients:

A method has been developed to evaluate protease activity in skin washings. Skin washings from patients with acute eczema may be obtained by aspirating 0.5 ml sterile physiological saline over the skin surface using a sterile, disposable plastic Pasteur pipette The skin area (˜1 cm²) is defined by a sterile open-ended plastic cylinder. Samples were transferred to 0.05 ml 0.55 M MOPS buffer pH 7.0, 55 mM calcium chloride and 0.2% Brij 35, mixed, centrifuged to remove debris and frozen at −70° C. pending analysis.

Zymographic analysis of the protease content of the samples may be done by mixing with 0.2 volume 0.2M Tris-HCl pH 6.8 containing 37.5% (v/v) glycerol and 2.5% sodium dodecylsulphate followed by electrophoresis through a gelatin zymogram gel (Invitrogen Corporation) according to the manufacturer's instructions. Gels were washed in 2.5% (w/v) Triton X-100 in 25 mM MOPS buffer pH 7 with or without compound 11 (50 μM) and developed overnight at 37° C. in 0.1M MOPS buffer pH 7 containing 5 mM calcium chloride with or without compound 11 (50 μM). Zones of clearing due to proteolytic activity may be identified by staining with Coomassie Brilliant Blue R followed by destaining in 40% (v/v) methanol/10% (v/v) acetic acid. Proteolytic activity may be attributed to aureolysin or metzincins by an appropriate method known to a person skilled in the art e.g. by molecular weight analysis with confirmation by Western blot.

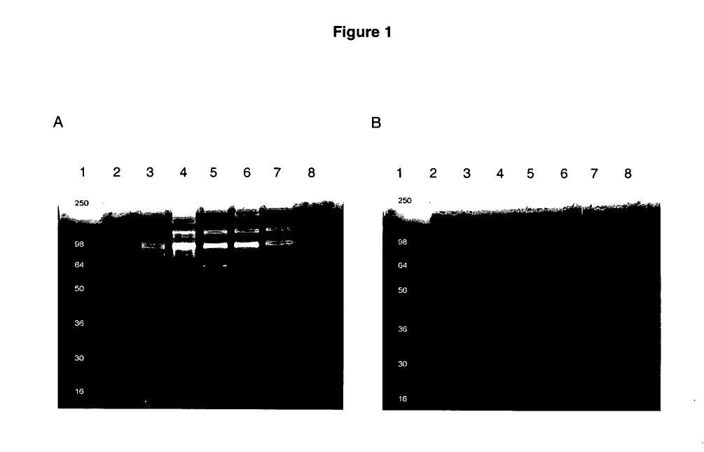

Representative gels obtained for 6 skin wash samples taken from sites of acute AD are shown in FIG. 1.

Legend to FIG. 1: (A) Zymographic analysis of 6 skin wash samples from patients with acute AD; (B) Zymographic analysis of the same skin wash samples incubated with 50 μM compound 11. For (A) and (B), lane 1=size markers (kDa); lane 2=skin wash sample 7; lane 3=sample 14; lane 4=sample 17; lane 5=sample 37; lane 6=sample 40; lane 7=sample 48; lane 8=4 ng purified aureolysin.

The protease activity in skin wash samples was also measured by incubating (9 μl) in 90 mM MOPS pH 7.0, 4.5 mM calcium chloride, 0.045% Brij 35, 10 μM Mca-Pro-Leu-Gly-Leu-Dap(Dnp)-Ala-Arg-amide (Bachem) and 2% (v/v) dimethyl sulphoxide vehicle with or without compound 11 (50 μM) at 37° C. Samples were incubated at 37° C. in a POLARstar Optima plate reader (BMG Labtech Ltd.) and fluorescence readings (320 nm excitation/405 nm emission) taken every 15 min for 6 h. Activity was expressed as the rate of increase in fluorescence as a function of time. Table 3 shows the results obtained.

TABLE 3

| | Protease | Protease activity with | % Inhibition protease |
|---|---|---|---|
| Sample | activity | 50 μM compound 11 | activity with compound |
| Number | (FIU/min/μl) | (FIU/min/μl) | 11 |
| 5 | 0.264 | 0 | 100 |
| 7 | 0.529 | 0.271 | 48.7 |
| 14 | 0.661 | 0.0761 | 88.5 |
| 17 | 3.42 | 0.123 | 96.4 |
| 18 | 0.276 | 0.0674 | 75.6 |
| 21 | 0.203 | 0.0302 | 85.1 |
| 24 | 0 | 0.00122 | N/A |
| 26 | 0.376 | 0.0554 | 85.3 |
| 30 | 0.226 | 0 | 100 |
| 36 | 0.366 | 0.0173 | 95.3 |
| 37 | 0.692 | 0.126 | 81.8 |
| 39 | 0.0564 | 0 | 100 |
| 40 | 0.716 | 0.103 | 85.6 |
| 43 | 0.994 | 0.0814 | 91.8 |
| 48 | 0.921 | 0.0287 | 96.9 |

Both assays show significant inhibition of protease activity in skin wash samples from patients with acute AD by compound 11.

Compound 12 was also tested for inhibition of protease activity in a set of 8 skin wash samples from AD patients. Table 4 below shows that there was significant inhibition of protease activity in the skin wash samples

TABLE 4

| | | Protease activity | % Inhibition |
|---|---|---|---|
| | | with 50 μM | protease activity |
| | Protease activity | compound 12 | with 50 μM |
| Sample number | (FIU/min/μl) | (FIU/min/μl) | compound 12 |
| 71-75 | 0.194 | 0.085 | 56 |
| 76-80 | 0.589 | 0.129 | 78 |
| 81-85 | 0.627 | 0.137 | 78 |
| 86-90 | 0.467 | 0.176 | 62 |
| 91-95 | 0.243 | 0.052 | 79 |
| 96-100 | 0.380 | 0.118 | 69 |
| 101-105 | 0.090 | 0.033 | 63 |
| 106-110 | 0.297 | 0.128 | 57 |

3. Staphylococcus Aureus Protease Activity in Culture.

Compound inhibition of metalloprotease (aureolysin) activity in Staphylococcus aureus 8325-4 culture supernatants was assessed in mixtures (0.1 ml) containing 90 mM MOPS buffer pH 6.8, 7.5 mM calcium chloride, 0.045% Brij 35, 10 μM Mca-Pro-Leu-Gly-Leu-Dap(Dnp)-Ala-Arg-amide (Bachem), 4 μl S. aureus culture supernatant and 2% (v/v) dimethyl sulphoxide vehicle with or without inhibitor. The S. aureus culture supernatant was prepared by inoculating 5 ml tryptic soy broth containing 10% skimmed milk with S. aureus 8325-4 and incubating at 37° C. for 6-8 h whilst shaking. The culture was then centrifuged to remove the cells and the supernatant stored at −70° C. for subsequent use. Reactions were incubated at 37° C. in a POLARstar Optima plate reader (BMG Labtech Ltd.) and fluorescence readings (320 nm excitation/405 nm emission) taken every 15 min for 6 h. Activity was expressed as the rate of increase in fluorescence as a function of time. The compound concentration eliciting a 50% decrease in enzyme activity under assay conditions (the IC₅₀ value) was determined by curve fitting (XLfit, IDBS Ltd). Table 5 shows the results obtained. These values are similar to the values obtained against purified aureolysin (Table 2) suggesting that the metalloprotease activity in supernatants is due to aureolysin activity.

TABLE 5

| Compound | IC₅₀ value (μM) |
|---|---|
| 1 | 0.57 |
| 3 | 20 |
| 5 | 44 |
| 7 | 4.5 |
| 9 | 2.9 |
| 11 | 3.7 |
| 12 | 4.3 |
| 13 | 0.69 |
| 14 | 3.6 |
| 15 | 3.3 |
| 16 | 0.095 |
| 17 | 2.3 |

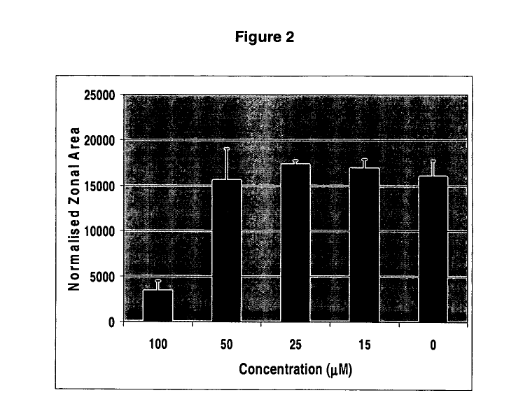

In a second assay for measuring S. aureus protease activity directly, S. aureus ATCC 27733 or 8325-4 was cultured on 10% (v/v) skimmed milk agar plates containing 2% (v/v) DMSO with or without compound. Compounds dissolved in DMSO were incorporated into the solid medium immediately prior to pouring. Agar plates were incubated at 37° C. for 24-48 hours and the proteolytic activity was assessed by measuring the zone of clearance around individual colonies. An example of this assay is shown in FIG. 2.

Legend to FIG. 2. The graph shows the inhibition of proteolytic activity by compound 3 in a milk agar plate assay. Results show zone of clearance of milk proteins.

4. Aureolysin-Mediated Protease Activation

The ability of aureolysin to activate endogenous proteases may be tested by incubating target protease with aureolysin in a suitable buffer containing calcium chloride, sodium chloride and Brij 35 at 37° C. This is exemplified below by the activation of pro-urokinase demonstrated directly by enzyme assay using a chromogenic substrate in the presence of EDTA to inhibit aureolysin activity (Narasaki et al J Biol. Chem. 240:14278-87, 2005) and by the activation of proMMP-1 demonstrated by measuring the production of the ¾-length cleavage product of the α1(I) chain of collagen using an appropriate imaging system.

The protease content of samples may also be determined using zymography by mixing with 0.2 volume 0.2M Tris-HCl pH 6.8 containing 37.5% (v/v) glycerol and 2.5% (w/v) SDS followed by electrophoresis through a gelatin zymogram gel (Invitrogen Corporation) according to the manufacturer's instructions. Zones of clearing due to proteolytic activity are identified by staining with Coomassie Brilliant Blue R followed by destaining in 40% (v/v) methanol/10% (v/v) acetic acid.

4(i). Activation of pro-uPA by Aureolysin and its Inhibition by Compound 13

The ability of aureolysin to activate urokinase-type plasminogen activator (uPA) was tested by incubating single chain pro-uPA (American Diagnostica Inc) with aureolysin at both physiological pH (7.5) and at pH 5.6, the natural pH of the stratum corneum (Ohman, H and Vahlquist, A Acta. Derm. Venereol. 74: 375-9, 1994). Incubation mixtures contained 1.4 μM (75 μg/ml) pro-uPA, 0.1M Tris-HCl pH 7.5 or 0.1M MES (sodium) buffer pH5.6, 0.1M sodium chloride, 5 mM calcium chloride, 0.05% Brij 35 and aureolysin in a final volume of 10 μl (Table 6, Expt. 1). In a second experiment activation at pH 5.6 was tested in the presence and absence of Compound 13 (20 μM) in a final volume of 20 μl (Table 6, Expt. 2). All samples were incubated at 37° C. for 2.5 h and then stopped by the addition of 24 volumes of 60 mM Tris-HCl pH 8.8, 50 mM sodium chloride, 2.5 mM EDTA, 0.01% Tween 80; this buffer also contained 0.83 μM Compound 13 when added to the vehicle control samples in Expt. 2. Urokinase activity was measured by incubating samples of the stopped mixture for 0.5 h at 37° C. in 0.1 ml of the same buffer containing 0.5 mM S-2444 (Chromogenix Instrumentation Laboratory SpA). The reactions were stopped with an equal volume of 0.5M acetic acid and the product measured at 405 nm.

Controls incubated in the absence of pro-uPA showed no activity in this assay.

TABLE 6

| Aureolysin (μg/ml) | (A₄₀₅/0.5 h/μg) | |
|---|---|---|
| Expt. 1 | MES pH 5.6 | Tris-HCl pH 7.5 |
| 0 | 0.14 | 0.19 |
| 0.1 | 2.31 | 0.65 |
| 0.3 | 3.76 | 1.43 |
| 1 | 4.68 | 3.29 |
| Expt. 2 | Compound vehicle only | Compound 13 (20 μM) |
| 0 | 0.13 | 0.11 |
| 0.1 | 2.37 | 0.36 |
| 0.3 | 4.15 | 0.52 |
| 0.8 | 5.29 | 0.96 |

The data in Table 6 show that: a) aureolysin activates pro-uPA at both pH 5.6 and 7.5; that b) this activation is more effective at the natural pH of the stratum corneum; and c) that Compound 13 at 20 μM (˜30× IC₅₀) inhibits this activity by ≧82%.

4(ii). Inhibition of pro-uPA Activation by Compound 12

The dose-response relationship for the inhibition of pro-uPA activation by Compound 12 was determined in reaction mixtures (20 μl) containing 1.5 μM (78 μg/ml) pro-uPA, 0.1M MES buffer (sodium) pH 5.6, 0.1M sodium chloride, 5 mM calcium chloride, 0.05% Brij 35, aureolysin (75 ng/ml) and 2% (v/v) DMSO±Compound 12. Mixtures were incubated at 37° C. for 2.5 h and stopped by dilution into 7 volumes of 60 mM Tris-HCl pH 8.8, 50 mM sodium chloride, 2.5 mM EDTA, 0.01% Tween 80. Under these conditions the extent of pro-uPA cleavage as assessed by uPA activity was directly proportional to the concentration of aureolysin in the assay. The uPA activity was measured using S-2444 as described at 4(i) above. The compound concentration eliciting a 50% decrease in uPA activity (the IC₅₀ value) was determined by curve fitting (XLfit, IDBS Ltd) to be 2.4 μM which is in good agreement with the value in Table 2 determined using a fluorogenic peptide substrate to assess aureolysin activity.

The ability of Compound 12 to inhibit the activation of pro-uPA at a higher aureolysin concentration was determined in reaction mixtures (20 μl) containing 1.5 μM (78 μg/ml) pro-uPA, 0.1M MES buffer (sodium) pH 5.6, 0.1M sodium chloride, 5 mM calcium chloride, 0.05% Brij 35, aureolysin (1 μg/ml) and 2% (v/v) DMSO±Compound 12. Mixtures were incubated at 37° C. for 2.5 h and stopped by dilution into 20 volumes of 60 mM Tris-HCl pH 8.8, 50 mM sodium chloride, 2.5 mM EDTA, 0.01% Tween 80 and uPA activity measured using. S-2444 as described at 4(i) above. The results in Table 7 confirm that, as expected, Compound 12 (at 10× and 130× IC₅₀ value) inhibits aureolysin-mediated pro-uPA activation in a dose-dependent manner. Compound 12 (317 μM) had no effect on uPA activity in this assay.

TABLE 7

| Aureolysin | Compound 12 | uPA activity | Relative activity |
|---|---|---|---|
| (μg/ml) | (μM) | (A₄₀₅/0.5 h/μg) | (%) |
| 0 | 0 | 0.17 | 4 |
| 1 | 0 | 4.2 | 100 |
| 1 | 25 | 1.6 | 37 |
| 1 | 317 | 0.3 | 8 |

Inhibition of the activation of pro-uPA by inhibiting aureolysin activity on the skin surface is expected reduce the pro-inflammatory drive in AD patients.

4(iii). Activation of proMMP-1 by Aureolysin

The ability of aureolysin to activate fibroblast collagenase (MMP-1) was tested by incubating the proenzyme of human rheumatoid synovial fibroblast collagenase (Calbiochem) at pH 7.5 with aureolysin both with and without the addition of the MMP activator aminophenylmercuric acetate (APMA).

Activation mixtures in TCNB buffer (0.1M Tris-HCl pH 7.5, 10 mM calcium chloride, 0.1M sodium chloride, 0.05% Brij 35) contained proMMP-1 (25 μg/ml) and aureolysin (3 μg/ml) and/or 1 mM APMA as indicated. Mixtures were incubated at 37° C./1.25 h and quenched by dilution into ice-cold TCNB. Collagenase activity in the samples was then determined by incubation at 25° C. in TCNB containing 0.1 μg/ml (pro)MMP-1 and 0.16 mg/ml porcine type I collagen (MD Biosciences). Portions of each mixture were removed at timed intervals into 0.2 volume 5× gel-loading buffer (0.2M Tris-HCl pH 6.8/37.5% (v/v) glycerol/2.5% (w/v) SDS/5% (v/v) 2-mercaptoethanol) and heated at 95° C. for 2.5 min. Collagen cleavage was quantified following SDS-PAGE gel analysis (4-12% NuPAGE Bis-Tris (MES), Invitrogen Corp) by estimating the band density of the ¾-length product of the α1(I) chain using a FluorChem™ 8800 imaging system running AlphaEase™ FC software (Alpha Innotech Corp.) Rates of cleavage were estimated from the linear portion of the curves and the rate relative to the untreated control calculated. The data are shown in Table 8 below. Consistent with the fact that aureolysin is not itself a collagenase, control incubations containing aureolysin alone±APMA showed no activity in this assay.

TABLE 8

| | α1-chain ¾ - length product (arbitrary units) | | | |
|---|---|---|---|---|
| | MMP-1 | MMP-1 + | MMP-1 + | MMP-1 + |
| Time (h) | alone | APMA | aureolysin | aureolysin + APMA |
| 0 | 0 | 0 | 0 | 0 |
| 0.5 | 1314 | 3387 | 1709 | 9652 |
| 1 | 1589 | 4871 | 3726 | 13867 |
| 1.5 | 1558 | 6404 | 5115 | 17022 |
| 2 | 1807 | 8115 | 8742 | 18227 |
| 3 | 3896 | 11474 | 12718 | 18435 |
| 4 | 6378 | 13850 | 16882 | 19988 |
| 5 | 8645 | 17154 | 17558 | 20211 |
| 6 | 10050 | 19387 | 19436 | 21388 |
| Relative | 1 | 2 | 3 | 8 |
| rate | | | | |

These data show that aureolysin not only activates proMMP-1 to an extent comparable with a recognised MMP-activator such as APMA but that it has the ability to “superactivate” MMP-1 when used in combination with APMA. Inhibition of aureolysin, therefore will inhibit proMMP-1 activation when the two enzymes are found at the same site, for example on the skin of patients with AD colonised with S. aureus.

5. Aureolysin-Mediated Keratinocyte Activation

Activated keratinocytes produce IL-8, a proinflammatory chemokine. Many bacterial products cause the activation of keratinocytes. Aureolysin may be evaluated for its effects on IL-8 production by keratinocytes. Human skin epidermal keratinocytes (TCS Cellworks) are maintained as per instructions. Proliferating cultures are trypsinised, harvested, treated with a trypsin inhibitor and resuspended in growth medium at approximately 50,000 cells/well, to provide confluent monolayers in 96 well plates. Cells are incubated overnight at 37° C. at 5% CO₂ to allow recovery, the spent medium aspirated from the wells and replaced with fresh growth medium. The cells are incubated at 37° C. at 5% CO₂ for a further 24 or 48 hours with aureolysin or buffer control.

The supernatants are removed from each well and the concentration of IL-8 is determined using a human IL-8 ELISA development kit from R&D systems (Catalog Number: DY208) using the manufacturers instructions.

Results of experiments are shown in Table 9. In these experiments, the cells are incubated at 37° C. at 5% CO₂ for 48 hours with aureolysin or buffer control. Poly IC and lipoteichoic acid (LTA), which stimulate IL-8 production in keratinocytes were used as positive controls.

TABLE 9

| Keratinocytes | IL-8 (pg/ml) ± SE |
|---|---|
| Unstimulated | 199 +/− 3.2 |
| Poly IC positive control | 282 +/− 0.9 |
| LTA positive control | 454 +/− 61.0 |
| Buffer control | 493 +/− 3.6 |
| Aureolysin (50 μg/ml) | 687 +/− 0.7 |

The experiment shows that aureolysin can stimulate IL-8 production in keratinocytes (687 pg/ml) over and above the aureolysin buffer control (493 pg/ml). LTA (454 pg/ml), and to a lesser extent, Poly IC (282 pg/ml) stimulated IL-8 production compared to the unstimulated control (199 pg/ml).

6. Impact of Compounds 11 and 12 on S. aureus Growth and Viability

The effect compounds on S. aureus growth and viability may be assessed by growing the organism in liquid culture followed by plating onto solid medium to count viable cells. Alternatively growth may be estimated by turbidometry in 96-well micro-titre plates.

Brain heart infusion medium (5 ml; Becton Dickinson and Co.) containing 10% skimmed milk and 1% (v/v) DMSO vehicle ±50 μM compound was inoculated with S. aureus 8325-4 (approximately 10⁷ cells in tryptic soy broth) and incubated for 16 h at 37° C./220 rpm. Duplicate samples (0.1 ml) were then removed from each culture, diluted into PBS, spread onto brain heart infusion agar (1.5%) and incubated at 37° C. Viable cell counts were determined from the number of colonies as shown in Table 10.

TABLE 10

| Compound | Cells/ml | sd |
|---|---|---|
| Vehicle | 1.3 × 10⁹ | 6.4 × 10⁸ |
| 11 | 1.6 × 10⁹ | 3.5 × 10⁸ |
| 12 | 1.3 × 10⁹ | 3.5 × 10⁸ |

Tryptic soy broth (0.18 ml) containing S. aureus 8325-4 (approximately 10⁵ cells) was mixed with 20 μl 20% (v/v) DMSO vehicle±compound in the wells of a flat-bottomed clear polystyrene 96-well micro-titre plate. The plate was incubated overnight at 37° C./220 rpm and the absorbance measured at 620 nm the following day. Growth inhibition was determined by reference to the vehicle control and the actinonin (Sigma) concentration eliciting a 50% decrease in terminal absorbance (IC₅₀ value) was determined by curve fitting (XLfit, IDBS Ltd) as shown in Table 11.

TABLE 11

| Compound | IC₅₀ value (μM) | Inhibition at 0.1 mM |
|---|---|---|
| Actinonin | 2.6 | — |
| Compound 11 | — | 2% |
| Compound 12 | — | 0% |

The data in Tables 10 and 11 shows that Compounds 11 and 12 are not anti-bacterial, whereas the positive control compound actinonin (Sigma), a hydroxamate-based peptide deformylase inhibitor (Clements, J M et al Antimicrob. Agents Chemother. 45:563-570, 2001), exhibits an apparent IC₅₀ value in this assay of 2.6 μM.

7. Impact of Compound 12 on S. aureus Protease Activities

Aureolysin is responsible for the activation of the staphylococcal serine protease glutamyl endopeptidase (V8 protease) and is indirectly responsible for the activation of the staphylococcal cysteine protease staphopain B (Shaw, L et al Microbiology 150:217-228, 2004). Inhibition of aureolysin would therefore be expected to have an impact on the activity of these proteases despite the fact that neither is a likely target of a metalloprotease inhibitor. The overall impact of compound on the activity of these staphylococcal proteases may be tested by growing S. aureus in the presence of compound and assaying the cell-conditioned medium for protease activity whilst maintaining the same concentration of that compound.

Duplicate samples of brain heart infusion medium (5 ml; Becton Dickinson and Co.) containing 10% skimmed milk and 2% (v/v) DMSO vehicle±Compound 12 were inoculated with S. aureus 8325-4 (approximately 10⁷ cells in tryptic soy broth) and incubated for 16 h at 37° C./220 rpm. Cultures were centrifuged to remove bacteria and the culture supernatants stored at −70° C. pending analysis of protease activity.

Enzyme activities were measured in mixtures containing 90 mM MOPS (sodium) buffer pH 7.0 (0.1 ml), 0.045% Brij 35, 2% (v/v) DMSO vehicle±Compound 12 and culture supernatant (4 μl). The concentration of compound used was the same as that which had been used to culture the assayed sample. Further additions to the reaction mixtures were as follows. Aureolysin assay: 4.5 mM calcium chloride, 9 μM E-64 (to inhibit cysteine protease activity) and 10 μM Mca-Pro-Leu-Gly-Leu-Dap(Dnp)-Ala-Arg-amide (Bachem); V8 protease assay: 10 μM Mca-Leu-Glu-Val-Asp-Gly-Trp-Lys(Dnp)-amide (Bachem); cysteine protease assay: 1.8 mM cysteine-HCl (pH-adjusted with NaOH), 9 mM EDTA and 0.1 mM Z-Phe-Arg-AMC hydrochloride (Bachem). The rate of product formation at 37° C. was monitored over 6 h taking readings at 15 min intervals using a POLARstar OPTIMA plate reader (BMG LABTECH Ltd) with 320 nm excitation/405 nm emission for aureolysin and V8 assays and 390 nm excitation/460 nm emission for the cysteine protease assay. Enzyme activities were determined from the linear rate of fluorescence increase and converted to percent inhibition by reference to vehicle control samples. The results in Table 12 show that Compound 12 almost completely suppresses the level of V8 protease activity and that it significantly suppresses the level of cysteine protease activity.

TABLE 12

| Compound 12 | | Inhibition (%) | |
|---|---|---|---|
| (μM) | Aureolysin | V8 protease | Cysteine protease |
| 317 | 99 | 99 | 87 |
| 159 | 98 | 97 | 78 |
| 79 | 94 | 91 | 53 |
| 40 | 89 | 83 | 27 |

The use of Compound 12 to inhibit aureolysin activity on the skin of AD patients colonised with S. aureus will therefore be expected to have the additional benefit of decreasing the activity of other extracellular staphylococcal proteases.

8. Selectivity Profile of Compound 12

Compound 12 has broad spectrum activity with comparable potency against aureolysin and MMPs (Table 2). Its inhibitory activity against other proteases, including those of different catalytic classes, may be determined by using suitably configured biochemical assays analogous to that used above for aureolysin. The inhibitory activity of Compounds 11 and 12 against a range of purified enzymes was tested as described below.

Inhibitory activity was tested in reaction mixtures (0.1 ml) containing 2% (v/v) DMSO vehicle±compound plus additions as follows. V8 protease: 90 mM MOPS (sodium) buffer pH 7.0, 435 mM calcium chloride, 0.045% Brij 35, 10 μM Mca-Leu-Glu-Val-Asp-Gly-Trp-Lys(Dnp)-amide (Bachem) and V8 (BioCentrum Ltd; 30 ng). Staphopain A and B: 90 mM MOPS (sodium) buffer pH 7.0, 1.8 mM cysteine-HCl (pH-adjusted with NaOH), 0.045% Brij 35, 0.1 mM Z-Phe-Arg-AMC hydrochloride (Bachem) and staphopain A or B (BioCentrum Ltd; 30 ng). Human kallikrein 5: 0.1M sodium phosphate buffer pH 8.0, 0.045% Brij 35, 0.1 mM Boc-Val-Pro-Arg-AMC (Sigma) and recombinant human kallikrein 5 (R&D Systems Inc; 6 ng). Human kallikrein 7: 72 mM Tris-HCl pH 8.0, 0.033% Brij 35, 1.2 mM S-2586 (Chromogenix Instrumentation Laboratory SpA) and recombinant human kallikrein 7 (R&D Systems Inc; 0.6 μg) that had been thermolysin-activated according to the manufacturer's instructions. Human angiotensin-converting enzyme (ACE): 45 mM MES (sodium) buffer pH 6.5, 0.045% Brij 35, 10 μM Mca-Arg-Pro-Pro-Gly-Phe-Ser-Ala-Phe-Lys(Dnp)-OH(R&D Systems Inc) and recombinant human ACE (R&D Systems Inc; 1.3 ng). Human cathepsin D: 0.1M sodium acetate buffer pH 3.5, 0.2M sodium chloride, 0.045% Brij 35, 10 μM Mca-Pro-Leu-Gly-Leu-Dap(Dnp)-Ala-Arg-amide (Bachem) and recombinant human cathepsin D (R&D Systems Inc; 8 ng) activated according to the manufacturer's instructions. Human ADAM17: 25 mM Tris-HCl pH 9.0, 2.5 μM zinc sulphate, 0.005% Brij 35, 10 μM Mca-Pro-Leu-Ala-Gln-Ala-Val-Dpa-Arg-Ser-Ser-Ser-Arg-amide (R&D Systems Inc) and recombinant human ADAM17 (R&D Systems Inc; 2.5 ng). All reactions were incubated at 37° C./1 h and stopped with 0.1 ml 0.5M acetic acid except for the cathepsin D assay which was stopped with 0.1 ml 0.15M Tris base. Percentage inhibition at 0.1 mM was calculated with reference to the vehicle control and, where appropriate, IC₅₀ values were determined by curve fitting (XLfit, IDBS Ltd). The data in Table 13 demonstrate that Compound 12 does not inhibit the aspartyl protease cathepsin D, the serine proteases kallikreins 5 and 7 and V8, nor the staphylococcal cysteine proteases staphopain A and B. Also, Compound 12 does not inhibit ACE (metalloprotease family M2) and it is only an extremely weak inhibitor of ADAM17 indicating that it is not a “sheddase” inhibitor; this contrasts markedly with its diastereoisomer, Compound 11, which is a potent inhibitor of ADAM17.

TABLE 13

IC₅₀ value or inhibition
at 0.1 mM

| Enzyme | Compound 11 | Compound 12 |
|---|---|---|
| ACE | — | 0% |
| ADAM17 | 0.025 μM | 79 μM |
| Cathepsin D | — | 1% |
| Kallikrein 5 | — | 0% |
| Kallikrein 7 | — | 0% |
| Staphopain A | 5% | 0% |
| Staphopain B | 11% | 10% |
| V8 protease | 0% | 0% |

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

### Abbreviations

ACE angiotensin-converting enzyme
AD atopic dermatitis
APMA 4-aminophenylmercuric acetate
DCC N,N′-dicyclohexylcarbodiimide
DCU N,N′-dicyclohexylurea
DMAP 4-dimethylaminopyridine
DMSO dimethylsulphoxide
E-64 L-trans-epoxysuccinyl-leucylamide-(4-guanidino)-butane
EDAC N-(3-dimethylaminopropyl)-N′-ethylcarbodiimide hydrochloride
EDC N-(3-dimethylaminopropyl)-N′-ethylcarbodiimide
EDTA ethylenediaminetetraacetic acid
HOBT 1-hydroxybenzotriazole hydrate
LTA lipoteichoic acid
MES 4-morpholineethanesulphonic acid
MOPS 4-morpholinepropanesulphonic acid
NMM N-methylmorpholine
PBS phosphate-buffered saline
TCNB 0.1M Tris-HCl pH 7.5, 10 mM CaCl₂, 0.1M NaCl, 0.05% Brij 35
TFA trifluoroacetic acid
THF tetrahydrofuran
uPA urokinase-type plasminogen activator

1. A method for the treatment or prevention of an inflammatory skin condition which is characterised by colonisation with Staphylococcus aureus, comprising the topical administration of an aureolysin inhibitor

2. A method according to claim 1, wherein the method is for the treatment or prevention of atopic dermatitis.

3. A method according to claim 1, wherein the aureolysin inhibitor is selected from known inhibitors of thermolysin.

4. A method according to claim 1, wherein the inhibitor of aureolysin is one which also inhibits one or more endogenous metzincin metalloproteases.

5. A method according to claim 1, wherein the aureolysin inhibitor is administered in combination with a further medicament.

6. A method according to claim 5, wherein the further medicament is an antibiotic.

7. A method according to claim 5, wherein the further medicament is one which modulates the inflammatory response, including steroidal and non-steroidal anti-inflammatory agents.

8. A method according to claim 5, wherein the further medicament is an immunosuppressant.

9. A method according to claim 1, wherein the aureolysin inhibitor does not significantly inhibit endogenous metzincin metalloproteases.

10. A method according to of claim 1, wherein the aurolysin inhibitor does significantly inhibit endogenous metzincin metalloproteases.

11. A method according to claim 1, wherein the aureolysin inhibitor is selected from Compounds 1-17 and pharmaceutically acceptable salts and solvates thereof.

12. A method according to claim 11, wherein the aureolysin inhibitor is Compound 12 or a pharmaceutically acceptable salt or solvate thereof.

13. A method according to claim 11, wherein the aureolysin inhibitor is Compound 16 or a pharmaceutically acceptable salt or solvate thereof.

14. A compound which is (R)—N1-((S)-3,3-dimethyl-1-(methylamino)-1-oxobutan-2-yl)-N4-hydroxy-2-isobutylsuccinamide (Compound 11) or a pharmaceutically acceptable salt or solvate thereof.

15. A compound which is (S)—N1-((S)-3,3-dimethyl-1-(methylamino)-1-oxobutan-2-yl)-N4-hydroxy-2-isobutylsuccinamide (Compound 12) or a pharmaceutically acceptable salt or solvate thereof.

16. A compound which is (S)—N4-hydroxy-2-isobutyl-N1-((S)-1-(methylamino)-1-oxo-4-phenylbutan-2-yl)succinamide (Compound 16) or a pharmaceutically acceptable salt or solvate thereof.

17. A compound which is (R)—N4-hydroxy-2-isobutyl-N1-((S)-1-(methylamino)-1-oxo-4-phenylbutan-2-yl)succinamide (Compound 17) or a pharmaceutically acceptable salt or solvate thereof.

18. A pharmaceutical composition comprising (S)—N1-((S)-3,3-dimethyl-1-(methylamino)-1-oxobutan-2-yl)-N4-hydroxy-2-isobutylsuccinamide (Compound 12) or a pharmaceutically acceptable salt or solvate thereof together with a pharmaceutically acceptable diluent or carrier.

19. A pharmaceutical composition comprising (S)—N4-hydroxy-2-isobutyl-N1-((S)-1-(methylamino)-1-oxo-4-phenylbutan-2-yl)succinamide (Compound 16) or a pharmaceutically acceptable salt or solvate thereof together with a pharmaceutically acceptable diluent or carrier.

20. A method of screening for an agent of use in the treatment or prevention of an inflammatory skin condition which is characterised by colonisation with Staphylococcus aureus, comprising:
(i) contacting said agent with aureolysin
(ii) determining if the aureolysin is inhibited

21. A method of screening for an agent of use in the treatment or prevention of an inflammatory skin condition which is characterised by colonisation with Staphylococcus aureus, comprising:
(i) obtaining skin washings from patients
(ii) contacting said agent with skin washings
(iii) determining whether proteolytic activity is inhibited

22. A method according to claim 21 wherein in step (iii) inhibition of proteolytic activity is determined by zymography or enzyme assay.

23. A method for the treatment of a skin lesion associated with an inflammatory skin condition in a mammal which is characterised by colonisation with Staphylococcus aureus which comprises (i) obtaining skin washings from the locus of said skin lesion and if the presence of metalloprotease activity is confirmed then (ii) topically administering an aureolysin inhibitor to said skin lesion.

24. A method for the treatment of a skin lesion associated with an inflammatory skin condition in a mammal which is characterised by colonisation with Staphylococcus aureus which comprises (i) determining the presence of Staphylococcus aureus in the locus of said skin lesion and if the presence of Staphylococcus aureus is confirmed then (ii) topically administering an aureolysin inhibitor to said skin lesion.

25. A method according to claim 24, wherein the aureolysin inhibitor is also an endogenous metzincin metalloprotease inhibitor.

26. A method or composition according to claim 1, wherein the aureolysin inhibitor is an aureolysin II inhibitor.
